(19) United States
(12) Patent Application Publication
(10) Pub. No.: US 2010/0034807 A1
(43) Pub. Date: Feb. 11, 2010

(54) METHODS AND COMPOSITIONS FOR DISCOVERY OF TARGET-SPECIFIC ANTIBODIES USING ANTIBODY REPERTOIRE ARRAY (ARA)

(76) Inventor: Matthew Moyle, Redmond, WA (US)

Correspondence Address: MINTZ, LEVIN, COHN, FERRIS, GLOVSKY AND POPEO, P.C, 5 Palo Alto Square - 6th Floor, 3000 El Camino Real, PALO ALTO, CA, 94306-2155, US

(21) Appl. No.: 12/509,323

(22) Filed: Jul. 24, 2009

Related U.S. Application Data
(63) Continuation of application No. 61/083,696, filed on Jul. 25, 2008.
(60) Provisional application No. 61/109,418, filed on Oct. 29, 2008.
(60) Provisional application No. 61/159,704, filed on Mar. 12, 2009.

(51) Int. Cl. A61K 39/395; C40B 50/06; C12P 21/00; C40B 30/04; G01N 33/53; C40B 40/10; A61P 31/12

(52) U.S. Cl. 424/130.1; 506/26; 435/68.1; 506/9; 435/7.2; 435/7.21; 506/18

(57) ABSTRACT

The invention provides antibody arrays specific for target antigens. Methods for discovery and compositions comprising native human antibodies, arrays comprising such antibodies, immortalized B cells expressing such antibodies and non-immortalized B cell libraries comprising B cells expressing such antibodies are provided. The invention provides a method for screening monoclonal antibodies for functional effects on cell surface molecules such as receptors using antibody repertoire arrays specific for target cell surface molecules. Functional antibodies directed to a target and therapeutics derived from such antibodies are also provided. High throughput and parallel screening for potentially therapeutic antibodies are provided. Antibodies directed to functional epitope clusters corresponding to a target and vaccines and therapeutics derived from such antibodies are also provided.

### CROSS-REFERENCE TO RELATED APPLICATIONS

This application claims priority of provisional patent applications U.S. Ser. No. 61/083,696, titled “Methods and Compositions for Discovery of Target-Specific Antibodies using Antibody Repertoire Arrays (ARA)” filed Jul. 25, 2008, U.S. Ser. No. 61/109,418, titled “Methods and Compositions for Discovery of Target-Specific Antibodies using Antibody Repertoire Arrays (ARA)” filed Oct. 29, 2008, and U.S. Ser. No. 61/159,704, titled “Function-Based Screening of Target-Specific Antibodies using Antibody Repertoire Arrays (ARA)” filed Mar. 12, 2009, the contents of which are incorporated herein in their entirety by reference.

### TECHNICAL FIELD OF THE INVENTION

This invention relates generally to antibody arrays specific for target antigens. Specifically, the invention relates to methods for discovery and compositions comprising native human antibodies, arrays comprising such antibodies and human B cells expressing such antibodies. The invention also relates to methods for high throughput and parallel screening for potentially therapeutic antibodies. The invention also relates to antibodies directed to functional epitope clusters corresponding to a target and vaccines and therapeutics derived from such antibodies.

### BACKGROUND OF THE INVENTION

Monoclonal antibodies that recognize extracellular domains of cell-surface receptors can act as agonists or antagonists of the receptors. Monoclonal antibody (MAb) 263 is a widely used monoclonal antibody that recognizes the extracellular domain (ECD) of the Growth Hormone (GH) receptor and shown to act as a GH agonist both in vitro and in vivo. (Wan Y., et al., Molecular Endocrinology 17 (11): 2240-2250 (2003)).

However, not all antibodies that bind a receptor appear to possess agonist or antagonist activity. Additional conformational changes may be required to elicit signaling. Not even all MAbs that are directed to the hormone binding site and act as full competitors for hormone binding are able to act as an agonist and elicit a signal. (Rowlinson S W, et al., 1998 J Biol Chem 273:5307-5314). A restriction in agonism to a narrow range of MAbs has also been reported for the erythropoietin receptor, where an extensive study showed that of 96 MAbs to the receptor, only four possessed agonist activity. (Elliott S, et al., 1996 J Biol Chem 271:24691-24697).

An antibody having agonist activity that stimulates a cell surface receptor might be an attractive therapeutic option in situations in which a prolonged half-life is needed and in which less frequent administration is desired. To gain insight into how a monoclonal antibody can activate a receptor, epitope mapping with known agonist MAbs have been employed. Further, mapping the binding site on the cell surface receptor of an agonist monoclonal antibody would improve our understanding of the structure function relationships of this receptor. A murine MAb, termed BAH-1, raised against human megakaryocytic cells that specifically recognizes the cell surface receptor (c-Mpl) for Thrombopoietin (TPO), shows agonist activity. (Deng B., et al., Blood, 92(6):1981-1988 (1998)).

Current methods for specifically detecting and quantifying a protein include antigen/antibody based-immunoassays. These assays include (a) classical direct immunoassays, such as immunodiffusion, immunoelectrophoresis, agglutination and immunoprecipitation assays, and (b) recently developed methods such as immunofluorescence, radioimmunoassay (RIA), enzyme-immunoassay (EIA) and Western blot assays. These approaches exploit the specificity of antigen-antibody interactions. However, they are designed for analyzing only one agent at a time, and are therefore limited as to the number of molecules that can be analyzed in a single assay.

A variety of display approaches are employed for the engineering of optimized human antibodies. Phage display is a widely used technology for the isolation of peptides and proteins with specific binding properties from large libraries of these molecules. Phage display of antibody libraries can be an alternative method for finding antibody fragments against targets. The use of phage display in screening for novel high-affinity ligands and their receptors has been crucial in functional genomics and proteomics. Display methods will make it possible to target essential components and pathways within many different diseases, including cancer, AIDS, cardiovascular disease, and autoimmune disorders.

Phage display is a widely used technology for the isolation of peptides and proteins with specific binding properties from large libraries of these molecules. Phage display of antibody libraries can be an alternative method for finding antibody fragments against targets. The use of phage display in screening for novel high-affinity ligands and their receptors has been crucial in functional genomics and proteomics. Display methods will make it possible to target essential components and pathways within many different diseases, including cancer, AIDS, cardiovascular disease, and autoimmune disorders.

A drawback of the common phagemid/helper phage systems is the high infective background of phages that do not display the protein of interest, but are propagated due to non-specific binding to selection targets. This and the enhanced growth rates of bacteria harboring aberrant phagemids not expressing recombinant proteins leads to a serious decrease in selection efficiency. The major drawback of the method when applied to antibodies is that the natural combination of light and heavy chains is lost and many false positive combinations are created. Therefore, the chance of finding the optimal combination of L and H chains (as developed by the natural immune system) is very low.

Affinity-matured antibodies expressed by human post-germinal center (post-GC) B-cells hold tremendous promise for the treatment of infectious diseases and bioterror exposures (Casadevall, A., Pirofski, L. A., 2005. Expert Opin. Biol. Ther. 5, 1359.). The best source for these antibodies may be individuals who have recovered from specific infections or vaccinations and have therefore produced definitive, protective antibody responses.

Native human antibodies are those that arise naturally as the result of the functioning of an intact human immune system. The utility of native antibodies for the treatment of human viral diseases has been established through experience with hyperimmune human globulins. One three-step method that uses human peripheral blood B-cells to produce stable hybridoma populations that are highly-enriched for affinity-matured human IgG antibodies against botulinum neurotoxins has been described. In this method, peripheral blood mononuclear cells (PBMCS) are (a) selected for expression of CD27, a marker of post-germinal center B-cells, (b) cultured in vitro to promote B-cell proliferation and class-switching and (c) fused to a genetically modified myeloma cell line. (Adekar et al., J Immunol Methods. 2008 Apr. 20; 333(1-2):156-66.).

Native antibodies, as a class, differ in some respects from those obtained by recombinant library methods (phage or transgenic mouse) and possess distinct properties that may make them ideal therapeutics for human diseases. (See Dessain et al., Exploring the Native Human Antibody Repertoire to Create Antiviral Therapeutics in Current Topics in Microbiology and Immunology 317: 155-183 (2008), © Springer-Verlag New York). Specifically, there is a specific advantage of libraries of native antibodies expressed from human B cells over phage-derived antibodies, due to the limitations in a phage approach to recreate all of the original or native heavy chain: light chain pairings, thus preventing important antibody structures from being incorporated into a phage-generated library. Therefore, it is desirable to obtain high-quality native human antibodies expressed from human B cells for detection, diagnosis, treatment and therapy of pathogens by a high-throughput method.

Further, immune recognition of every potential epitope derived from a pathogen's genome may not be required. Response to a subset of antigens and epitopes derived from an infectious pathogen may be sufficient for competent protection. Thus “immunome-derived antivirals” are based on the concept that response to the subset of antigens and epitopes that interface with the host immune system (the immunome) and not the whole organism (represented by the proteome or genome) can be sufficient for protection. Competent immune responses to cancer are also probably restricted to the immunome provoked by the neoplasm. Therefore, it is desirable to obtain an antibody library comprising the human immunome relating to any given infection or neoplasm.

Another drawback of current methods for screening antibody libraries is that the information obtained is based essentially on abilities to bind a target and provide little or no screening based on the functional effects of the antibody when bound to a target. The late nineties saw an explosion in the area of genomic and proteomic technology, promising to uncover a whole set of novel targets. High throughput screening and computer-aided analyses of nucleotide and protein arrays from normal and tumor tissues revealed a world of subtle differences at protein level which could theoretically be targeted for cancer therapy. However, none of these have led to a clinically useful and validated target. Subtle differences in expression patterns may not be as important and tumor-selective function may be more relevant. Investigation of new targets to establish which ones make a functional difference to tumor cells could be at the level of epitope distinction, where binding to one epitope leads to an alteration in signaling whereas binding to another epitope has a different property. This would allow combination therapy or second-line therapy with antibodies that block different functions and act synergistically, in situations where monotherapy regimens have lost their effectiveness.

For example, TRASTUZUMAB® (HERCEPTIN®; Genentech, San Francisco, Calif.) is a recombinant humanized monoclonal antibody directed against the extracellular domain of the HER-2 (Human Epidermal growth factor Receptor 2; erb-B2; neu) tyrosine kinase receptor. Clinical studies established that TRASTUZUMAB® is active against HER-2-overexpressing metastatic breast cancers, leading to its approval in 1998 by the United States Food and Drug Administration (Carter P, Presta L, Gorman C M, et al. Humanization of an anti-p185her2 antibody for human cancer therapy. Proc Natl Acad Sci USA (1992) 89:4285-4289.). Another HER-2-targeted monoclonal antibody, PERTUZUMAB® (OMNITARG®, 2C4; Genentech), is currently being tested in Phase I clinical trials in cancer patients with different types of solid tumors. In contrast to TRASTUZUMAB®, PERTUZUMAB® functions differently by sterically blocking HER-2 dimerization with other HER receptors and blocks ligand-activated signaling from HER-2/EGFR and HER-2/HER-3 heterodimers (Agus D B, et al. Cancer Cell (2002) 2: 127-137.). As the majority of breast tumors that initially respond to TRASTUZUMAB® begin to progress again within 1 year (Cobleigh M A, et al. J Clin Oncol 1999; 17:2639-2648), treatment with combined TRASTUZUMAB® and PERTUZUMAB® have been found to synergistically block the survival of HER-2-overexpressing BT474 breast cancer cells. (Nahta R., et al. Cancer Res. 64, 2343-2346 (2004)). Therefore, it is desirable to obtain an antibody library comprising groups of antibodies classified by functional or epitope specific properties in a rapid and high-throughput manner.

The erbB2 oncogene encodes a growth factor receptor. The overexpression of erbB2 has been correlated with more aggressive tumors and a poorer prognosis. Some antibodies directed to this molecule have an antitumor effect in vivo, but some antibodies do not. (Wang et al. Mol Immunol 2004 February; 40(13):963-969). Evidently, some epitopes correspond to tumor growth-related functions of erbB2 while others do not. Therefore, there is a need for comprehensive exploration of epitope space within a given target.

### SUMMARY OF THE INVENTION

The following description of various embodiments of methods, compositions, and kits is not to be construed in any way as limiting the subject matter of the appended claims.

The present invention relates to methods for discovery of native human antibodies that should facilitate the creation of novel, potent therapeutics, diagnostics and prognostics obtained from the native human antibody repertoire. The present invention provides methods for producing a library comprising antibodies in their native human configuration. The present invention further provides a novel antibody repertoire array (ARA) comprising antibodies from the library of native human antibodies for the discovery of native human antibodies targeted against specific antigens. The invention provides novel compositions and kits comprising native human antibodies targeted against specific antigens, discovered by use of the ARAs of the invention.

The present invention relates to a method for rapidly identifying monoclonal antibodies that possess a specific function from a pool of monoclonal antibodies that are directed against a specific target cell surface molecule, such as a receptor. The invention provides novel compositions and kits comprising native human antibodies targeted against specific antigens and having specific functions that are discovered by use of a target-specific antibody repertoire array (ARA) of the invention.

The present invention relates to a method for screening monoclonal antibodies for the presence of a biological function the method comprising: providing an antibody repertoire array (ARA) comprising a plurality of monoclonal antibodies directed against a specific target molecule that is present on a cell surface; contacting the ARA with cells comprising the specific target molecule that is present on the cell surface; and identifying those monoclonal antibodies which have an inhibiting or activating effect on the specific target molecule that is present on the cell surface.

The method may further comprise: contacting the ARA with reporter cells, wherein the reporter cells have been engineered to express a detectable signal when contacted with an agonist or antagonist of the cell surface target molecule present on the surface of the reporter cell; and incubating the reporter cell with the monoclonal antibodies in the presence of a substrate necessary for generating a detectable signal, wherein a change in level of the detectable signal indicates the presence of a cell surface target molecule antagonist or agonist function of the monoclonal antibody.

In some aspects, the specific target molecule that is present on the cell surface is a receptor molecule.

In some aspects, the receptor is selected from the group consisting of: peripheral membrane protein receptors, transmembrane receptors, metabotropic receptors, G protein-coupled receptors (GPCRs), receptor tyrosine kinases, guanylyl cyclase receptors, ionotropic receptors responsive to extracellular ligands, receptor tyrosine kinases, cytokine receptors, receptor guanylyl cyclases, receptor serine/threonine protein kinases, insulin receptor, insulin-like growth factor receptor, human growth hormone receptor, glucose transporters, transferrin receptor, epidermal growth factor receptor, low density lipoprotein receptor, leptin receptor, interleukin receptors, IL-1 receptor, IL-2 receptor, GPCRs, muscarinic acetylcholine receptor, adenosine receptors, adrenoceptors, gaba receptors, angiotensin receptors, cannabinoid receptors, cholecystokinin receptors, dopamine receptor, glucagon receptors, metabotropic glutamate receptors, histamine receptors, olfactory receptors, opioid receptors, rhodopsin, secretin receptors, serotonin receptors, somatostatin receptors, calcium-sensing receptors, growth factor receptors, co-stimulatory factor receptors, protease-activated receptors, T cell receptors, B cell receptors, ITIM-containing receptors, ITAM-containing receptors, members of the TNFR superfamily, members of the TNF superfamily, ion channels, and chemokine receptors.

In some aspects, the antibody functions as a full agonist, partial agonist, antagonist or inverse agonist of the receptor protein.

In some embodiments the detectable signal is fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, enzyme that yields a magnetic product or ruthenium.

The present invention relates to a screening method wherein the activation of the cell surface molecule is coupled to an intracellular signaling pathway linked to an activity of an enzyme capable of effecting a substrate.

In some embodiments the enzyme is selected from the group consisting of β-lactamase, α-galactosidase, β-galactosidase, α-glucosidase, β-glucosidase, α-mannosidase, β-mannosidase, acid phosphatase, alkaline phosphatase and phosphodiesterase II.

In some embodiments the substrate is selected from the group consisting of p-aminophenyl-β-D-galactopyranoside, p-aminophenyl-α-D-galactopyranoside, p-aminophenyl-α-D-glucopyranoside, p-aminophenyl-β-D-glucopyranoside, p-aminophenyl-α-D-mannopyranoside, p-aminophenyl-β-D-mannopyranoside, p-aminophenylphosphate, and p-aminophenylphosphorylcholine or derivative thereof.

In some embodiments the effect of the enzyme on the substrate is couple to a chemical, luminometric, calorimetric or fluorimetric reaction.

The present invention relates to a screening method further comprising: removing unbound reporter cells from the surface of the ARA with a fluid shear force prior to detection of antibody function.

In some aspects, the ARA is arranged in a 96 or 384 well plate. Each well comprises monoclonal antibodies from a single B cell clone, wherein the concentration of monoclonal antibodies is sufficient to elicit a signal from the cell surface target molecule. Each well is contacted with greater than 10³ reporter cells. In some embodiments, each well is contacted with less than 10³ reporter cells and cell growth is permitted under suitable conditions until 10³ or more reporter cells are present in each well before the detectable label is observed.

In some aspects, the detectable label which may or may not be secreted from the reporter cell is detected within the well in which it is generated.

In some aspects, each well is contacted with reporter cells which are incubated under conditions suitable for cell growth until a concentration in the order of 10³, 10⁴, 10⁵ or more reporter cells is reached. The cell growth conditions are suitable also for expression of the detectable label.

In some aspects, the screening is a high throughput screen. In some aspects, the screening is a high-content screen.

In some aspects, the detectable label is generated indirectly from the activation of the cell surface target molecule.

In some aspects, activation of the cell-surface target molecule signaling pathway is coupled to β-lactamase expression and expression of β-lactamase is quantified using a fluorescence resonance energy transfer (FRET)-based substrate.

The invention provides a method for producing an antibody library, comprising: obtaining at least 10⁴ B-cells from each of an effective number of human donors, and forming a population of B-cells, wherein said population contains at least 10⁵, preferably at least 10⁶, more preferably at least 10⁷ different species of naturally occurring antibodies wherein each of the antibodies has naturally paired heavy and light chains representing substantially the entire human immunome; dividing said population of B cells into subpopulations of B cells wherein each subpopulation produces on average 1, 5, 10, 20, 50 or 100 different species of antibodies; expanding each subpopulation of B cells to produce an expanded B-cell culture; optionally immortalizing each of said B-cell cultures prior to or following expansion to produce an immortalized B-cell culture; culturing each of said B-cell cultures in culture medium under condition in which said B-cells secrete antibodies into said culture medium; and attaching or disposing each of said antibodies at distinct locations on a solid surface, thereby producing an antibody array.

In a further step, the method comprises identifying an antibody that is specific for said target. The method may further comprise the steps of determining which immortalized or non-immortalized B-cell culture produced said target antibody; and isolating the B-cell producing said target antibody from said B-cell culture.

The invention provides a method for producing antibodies from one or more individual donors comprising: obtaining at least 10⁴ B-cells from said one or more donors with naturally expressed antibodies; dividing said B-cells into subpopulations producing at least 1 species of antibody, preferably subpopulations producing about 1-100 antibodies; expanding each subpopulation of B cells to produce an expanded B-cell culture; optionally immortalizing each of said B-cell cultures prior to or following expansion to produce an immortalized B-cell culture; culturing each of said B-cell cultures in culture medium under condition in which said B-cells secrete antibodies into said culture medium; and attaching said antibodies at distinct locations on a solid surface. The method may further comprise the step of: screening said antibodies against a target.

The invention provides methods wherein said number of human donors is at least 10, 50, 100 or 500.

The invention provides methods wherein said population of B-cells is divided into at least 10, 20, 50, 100, 1000, 10⁴ and up to 10⁷ subpopulations.

The invention provides an antibody library comprising at least 10⁵, preferably at least 10⁶, more preferably at least 10⁷, naturally occurring antibodies having naturally paired V_{H} and V_{L} regions, wherein said antibodies have been expressed from human B-cells, preferably immortalized human B cells, that were obtained from a sufficiently diverse patient population such that the antibodies in said library have a diversity of binding activities substantially similar to the entire human immunome.

The invention provides an array and an antibody library comprising at least 10⁵, preferably at least 10⁶, more preferably 10⁷ or greater naturally expressed human native antibodies having naturally paired V_{H} and V_{L} regions, wherein said antibodies have been expressed from human B-cells. In some embodiments, the antibody library or ARA recognizes at least 10⁵ different unique antigens or targets, preferably at least 10⁶, and more preferably 10⁷ or greater different unique antigens or targets. See, e.g., U.S. Pat. No. 6,319,690, fully incorporated herein by reference.

The invention provides a library comprising a population of human B cells producing at least 10⁵, preferably at least 10⁶, and more preferably 10⁷ or greater different species of naturally occurring antibodies wherein each of the antibodies has naturally paired V_{H} and V_{L} regions, wherein the population of human B cells is divided into subpopulations of B cells wherein each subpopulation produces on average 1-100 different species of antibodies, and wherein said human B-cells were obtained from a sufficiently diverse patient population such that the antibodies produced by the B cells in said library have a diversity of binding activities substantially similar to the entire human immunome.

The invention provides a method for producing a non-immortalized B-cell library comprising: obtaining at least 10⁴ memory B-cells from each of an effective number of human donors; preparing a population of human B-cells, wherein said population contains at least 10⁵, preferably at least 10⁶, and more preferably 10⁷ or greater different species of naturally occurring antibodies wherein each of the antibodies has naturally paired heavy and light regions; dividing said population of B cells into subpopulations of B cells wherein each subpopulation produces on average 1-100 different species of antibodies; optionally, expanding each subpopulation of B cells to produce an expanded B-cell culture; and storing each sub-population under conditions suitable for preserving its RNA content, wherein a library of non-immortalized B-cell populations each expressing on average 1-100 different species of antibodies is produced. The method may further comprise the steps of: preparing RNA samples corresponding to each stored sub-population of B-cells; performing reverse transcriptase-polymerase chain reaction (RT-PCR) on each RNA sample; isolating DNA corresponding to V_{H} and V_{L} regions capable of natural pairing; cloning said DNA corresponding to V_{H} and V_{L} regions in a suitable host capable of expression of said V_{H} and V_{L} regions; and expressing said V_{H} and V_{L} regions in the context of an immunoglobulin heavy and light chain, such that a naturally paired immunoglobulin (Ig) is formed.

The invention provides a method for isolating target specific antibodies, comprising: obtaining B-cells from human donors previously exposed to the target, wherein said B-cell population contains at least 10⁵ different species of naturally occurring antibodies with naturally paired heavy and light chains; dividing said population of B cells into subpopulations of B cells wherein each subpopulation produces on average 1-100 different species of antibodies; expanding each subpopulation of B cells to produce expanded B-cell cultures under conditions in which said B-cells secrete antibodies into said culture medium; disposing said antibodies secreted into the culture medium from each of said B-cell cultures at distinct locations on a solid surface to create an antibody repertoire array (ARA); interrogating the antibody repertoire array with a native target molecule to identify one or more antibody populations that is specific for said target; preparing RNA samples from each of said B-cell cultures corresponding to an antibody populations that is specific for said target; performing reverse transcriptase-polymerase chain reaction (RT-PCR) on a plurality of the RNA samples; isolating DNA corresponding to V_{H} and V_{L} regions capable of natural pairing; cloning said DNA corresponding to V_{H} and V_{L} regions in a suitable host capable of expression of said V_{H} and V_{L} regions; and expressing said V_{H} and V_{L} regions in the context of an immunoglobulin heavy and light chain, such that a naturally paired immunoglobulin is formed. In some embodiments the target is a virus, bacteria, an yeast, a parasite, a fungus, or other pathogen. In some embodiments, the native target molecule is a virion, a virus like particle, a virus infected cell, or a viral protein. In one embodiment, the target is human immunodeficiency virus (HIV).

In one aspect the method further comprises providing a plurality of targets comprising multiple species of targets or a plurality of serotypes of the same target; and identifying cross-reactive antibodies.

The invention provides antibody repertoire arrays (ARA) prepared by any method described herein.

The invention provides a method for screening antibodies based on epitope clustering, the method comprising: providing a gene fragment phage display (GFPD) library generated from gene fragments representing parts of a target protein, wherein the GFPD library members are clustered according to correspondence with one or more epitopes; providing an intact target protein; providing an antibody repertoire array (ARA) generated from blood samples of subjects with prior exposure to amounts of a target sufficient to mount an immune response; interrogating the ARA with the intact target and epitope-specific clusters of GFPD library members derived from the target; identifying one or more antibody populations that is specific for said intact target and at least one epitope cluster; preparing RNA samples from each of said B-cell cultures corresponding to an antibody population that is specific for said epitope cluster; performing reverse transcriptase-polymerase chain reaction (RT-PCR) on a plurality of the RNA samples; isolating DNA corresponding to V_{H} and V_{L} regions capable of natural pairing; cloning said DNA corresponding to V_{H} and V_{L} regions in a suitable host capable of expression of said V_{H} and V_{L} regions; and expressing said V_{H} and V_{L} regions in the context of an immunoglobulin heavy and light chain, such that a naturally paired immunoglobulin is formed.

In one aspect the method further comprises identifying a new epitope based on the pattern of recognition of the ARA by the intact target and the GFPD library members. The method comprises the additional steps of preparing RNA samples from each of said B-cell cultures corresponding to an antibody population that is specific for a new epitope cluster; performing reverse transcriptase-polymerase chain reaction (RT-PCR) on a plurality of the RNA samples; isolating DNA corresponding to V_{H} and V_{L} regions capable of natural pairing; cloning said DNA corresponding to V_{H} and V_{L} regions in a suitable host capable of expression of said V_{H} and V_{L} regions; and expressing said V_{H} and V_{L} regions in the context of an immunoglobulin heavy and light chain, such that a naturally paired immunoglobulin is formed.

The invention relates to a therapeutic antibody prepared by expressing identified and cloned V_{H} and V_{L} chain according to the methods described herein.

The invention relates to a method of preparing a gene fragment phage display (GFPD) library, wherein the GFPD members are clustered according to correspondence with one or more epitopes, is done by a method comprising: providing a gene encoding a target protein; fragmenting said gene into gene fragments; preparing a phage display library comprising the GFPD library members; panning the GFPD library on human antibodies specific for the target; and grouping each GFPD according to correspondence with one or more clusters.

The methods further comprise grouping GFPD library members overlaying the GFPD library members on a known three dimensional structure of the target, wherein a function of the target is associated with a portion of the known three dimensional structure of the target.

The invention relates to a method for testing synergistic function between two or more epitope clusters identified by the methods described herein, the method comprising: preparing a first naturally paired immunoglobulin formed by expressing V_{H} and V_{L} regions sequenced from an antibody population that is specific for an epitope cluster; preparing a second naturally paired immunoglobulin formed by expressing V_{H} and V_{L} regions sequenced from an antibody population that is specific for a different epitope cluster; administering both first and second naturally paired immunoglobulins individually and in combination to a test system for measuring activity of the intact target; and determining an activity or a synergy of activities of the new epitope that is related to the known function.

The invention provides a small molecule and a therapeutic antibody preparation that is effective in modulating a function of the target associated with one or more epitope clusters determined by the methods described herein. The invention provides a vaccine preparation, comprising antibodies effective against a functional epitope cluster determined by the methods described herein.

The invention provides a kit comprising a therapeutic antibody capable of altering a function of a cell surface receptor.

The invention provides a kit for screening monoclonal antibodies having a specific function, the kit comprising: an antibody repertoire array (ARA) comprising a plurality of monoclonal antibodies directed against a specific target molecule that is present on a cell surface; and optionally, reporter cells, wherein the reporter cells have been engineered to express a detectable signal when contacted with an agonist or antagonist of the cell surface target molecule present on the surface of the reporter cell.

The present invention and other objects, features, and advantages of the present invention will become further apparent in the following Detailed Description of the invention and the accompanying figures and embodiments.

### BRIEF DESCRIPTION OF THE FIGURES

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1 shows a schematic diagram of an antibody discovery process using antibody repertoire arrays.

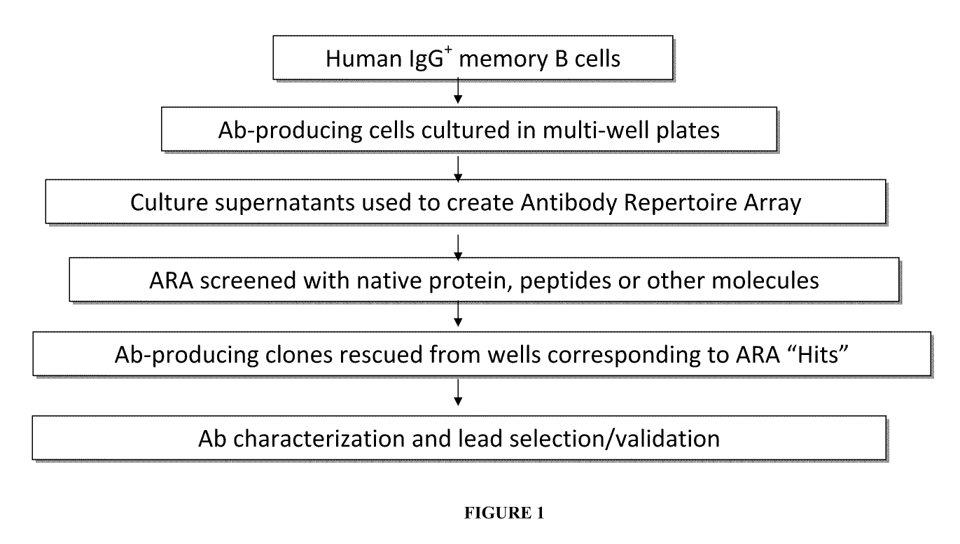

FIG. 2 shows a schematic diagram of a process for discovery of monoclonal antibodies against HIV using the ARA platform.

FIG. 3 shows a schematic diagram of a process for generating a phage display of the epitope repertoire corresponding to a human gene.

FIG. 4 shows a schematic diagram of a process for screening an ARA with whole protein or pathogen as targets and also with individual epitopes as targets.

FIG. 5 shows a schematic diagram of a process for isolating unique antibody clusters directed to individual functional epitopes on a target.

### DETAILED DESCRIPTION OF THE INVENTION

Without further elaboration, it is believed that one skilled in the art can, using the following description, utilize the present invention to its fullest extent. The following description is illustrative only, and not limiting of the remainder of the disclosure in any way whatsoever.

The utility and need for passive antibody therapy for treatment of infectious diseases have been recognized. (Keller and Stiehm. Clin. Microbiol. Rev. 13:602-614 (2000); Oral H B et al. Mol. Biotechnol. 21:225-239 (2002); Casadevall et al. Nat. Rev. Microbiol. 2:695-703 (2004). Individuals who have recovered from a viral infection or who have received a therapeutic vaccination contain populations of antibodies that contribute to life-long immunity from the virus. These “native antibodies” have the heavy and light chains paired in exactly the same configuration as created by a functioning human immune system. They are distinct from human or humanized antibodies created from recombinant systems or using transgenic mouse systems in that these do not replicate the “wild-type” structure of full length antibodies created naturally by a human system.

The native human antibody repertoire holds unexplored potential for the development of novel monoclonal antibody therapeutics. The native human antibody repertoire contains definitive immunological solutions to human diseases and is likely to be the safest for human clinical use. While polyclonal antibody therapeutics using intravenous immunoglobulins (IVIG; using native IgG from plasma) have been used in the past, the present invention relates to novel methods for exploiting with far greater efficiency, the therapeutic potential of cloned native human antibodies. Antibody libraries or arrays have been constructed (see, e.g., U.S. Pat. Nos. 4,829,010 and 4,591,570, both fully incorporated by reference); however there has been no library or ARA of human native antibodies that comprise substantially all of the human native immunome, as described and claimed here.

The present invention provides an antibody repertoire array (ARA) for antibody discovery. In one aspect a high-throughput, multiplexed and scalable platform for the comprehensive interrogation of the antibody repertoire of a given donor or pool of donors is provided. In one aspect the invention provides a large candidate pool to increase the probability of identifying a high-quality antibody with unique functional properties.

The present invention relates to a method for rapidly identifying antibodies that possess a specific function from a pool of monoclonal antibodies provided in an antibody repertoire array (ARA). In one aspect a high-throughput, multiplexed and scalable platform for the comprehensive interrogation of the antibody repertoire of a given donor or pool of donors is provided. In one aspect the invention provides a large candidate pool to increase the probability of identifying a high-quality antibody with unique functional properties.

A receptor is a protein molecule, embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, a toxin, or an antibody and when such binding to an agonist occurs, the receptor goes into a conformational change which ordinarily initiates a cellular response. Some ligands (e.g. antagonists) merely block receptors without inducing any response. Ligand-induced changes in receptors result in physiological changes which constitute the biological activity of the ligands.

Receptors according to the invention include peripheral membrane protein receptors, transmembrane receptors, metabotropic receptors, G protein-coupled receptors (GPCRs), receptor tyrosine kinases, guanylyl cyclase receptors, ionotropic receptors responsive to extracellular ligands and the like. Transmembrane proteins may contain from one to many transmembrane domains. For example, receptor tyrosine kinases, certain cytokine receptors, receptor guanylyl cyclases and receptor serine/threonine protein kinases contain a single transmembrane domain. However, various other proteins including ion channels and adenylyl cyclases contain numerous transmembrane domains. Many important cell surface receptors are classified as “seven transmembrane domain” (7™) proteins, as they contain 7 membrane spanning regions. Important transmembrane protein receptors include, but are not limited to insulin receptor, insulin-like growth factor receptor, human growth hormone receptor, glucose transporters, transferrin receptor, epidermal growth factor receptor, low density lipoprotein receptor, leptin receptor, interleukin receptors, e.g. IL-1 receptor, IL-2 receptor, etc. GPCRs include muscarinic acetylcholine receptor, adenosine receptors, adrenoceptors (also known as adrenergic receptors), GABA receptors, angiotensin receptors, cannabinoid receptors, cholecystokinin receptors, dopamine receptor, glucagon receptors, metabotropic glutamate receptors, histamine receptors, olfactory receptors, opioid receptors, rhodopsin, secretin receptors, serotonin receptors, somatostatin receptors, calcium-sensing receptor, chemokine receptors, cytokine receptors and the like. Certain receptors are involved in signal transduction.

Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted.

The extracellular domains of transmembrane proteins are diverse; however, conserved motifs are found repeatedly among various extracellular domains. Conserved structure and/or functions have been ascribed to different extracellular motifs. For example, cytokine receptors are characterized by a cluster of cysteines and a WSXWS (W=tryptophan, S=serine, X=any amino acid) motif. Immunoglobulin-like domains are highly conserved. Mucin-like domains may be involved in cell adhesion and leucine-rich repeats participate in protein-protein interactions.

Many extracellular domains are involved in binding to other molecules. In one aspect, extracellular domains are receptors. Factors that bind the receptor domain include circulating ligands, which may be peptides, proteins, or small molecules such as adenosine and the like. For example, growth factors such as EGF, FGF and PDGF are circulating growth factors that bind to their cognate receptors to initiate a variety of cellular responses. Other factors include cytokines, mitogenic factors, neurotrophic factors and the like.

According to the present invention, a functional monoclonal antibody interacts with an extracellular domain of a cell surface protein and elicits a biological response, directly or indirectly.

Agonists are able to activate the receptor and result in a maximal biological response. Most natural ligands are full agonists. Partial agonists do not activate receptors thoroughly, causing responses which are partial compared to those of full agonists.

Antagonists bind to receptors but do not activate them. This results in receptor blockage, inhibiting the binding of other agonists. Inverse agonists reduce the activity of receptors by inhibiting their constitutive activity. A monoclonal antibody that binds to a receptor can have any one or more of these effects.

The invention enables sensitive detection of very rare antibodies (one in 10⁵-10⁶) in individuals or donor populations. Methods for identifying and confirming target specific native human antibody populations in a rapid time frame (3 months or less) are provided.

Further, methods of the invention allow interrogation of target molecules in native configuration with native human antibodies with natural light and heavy chain pairing resulting in a screen that identifies high quality antibodies against a specific target.

Human IGG+ Memory B Cells from Human Donors

The human immune system comprises 10¹² B-cell clonotypes in an individual with over 10⁹ combinatorial antibodies (Jerne N K, Scand J. Immunol. 38(1):1-9 (1993)). However, as used herein, a population of B cells containing at least 10⁵, preferably at least 10⁶, more preferably at least 10⁷ different species of IgG antibodies is considered to be representative of a human native immunome responsive to antigen(s) corresponding to a disease, disorder or infectious agent. From each donor, at least 10⁴ B-cells are collected. The human native antibody library and array contemplated here comprises substantially all of the possible native human antibodies that can be created by a functioning, intact human immune system in response to responsive to antigen(s) corresponding to a disease, disorder or infectious agent, and typically contains at least 10⁵, preferably at least 10⁶, more preferably at least about 10⁷ different species of human native antibodies, collected from at least 10 different donors.

Intravenous immunoglobulins (IVIG) comprise a purified population of native human IgG antibodies obtained from blood plasma and reflects the collective antibody immunome of the population from which it is generated. It has been observed that geographically different donor pools differ in titers of specific antibodies. Therefore, in one aspect of the invention, the donor pool is generated from a geographically diverse population to enhance the diversity of target-specific antibodies.

In one aspect of the invention, the donor population is untreated or has not been subject to an infection with a common infectious agent or been subject to a common therapeutic vaccination.

In another aspect of the invention, where antibodies targeted to a specific infectious agent or human disease is desired the donor pool is selected for a population that suffers from a common ailment, or has been infected by or vaccinated against a common infectious agent.

In one embodiment, the donors are known to have developed a target disease, such as at least one disease from infectious disorders such as influenza viral infection, hepatitis C virus (HCV) infection, herpes simplex virus (HSV) infection, human immunodeficiency virus (HIV) infection, Methicillin-resistant Staphylococcus aureus (MRSA) infection, Epstein-Barr virus (EBV) infection, respiratory syncytial virus (RSV) infection, Pseudomonas, Candida infections; respiratory disorders such as asthma, allergies, chronic obstructive pulmonary disease (COPD), idiopathic pulmonary fibrosis (IPF), adult respiratory distress syndrome (ARDS), metabolic disorders such as frailty, cachexia, sarcopenia, obesity, dyslipidemia, metabolic syndrome, myocardial infarction (MI), chronic renal failure (CRF), osteoporosis digestive disorder irritable bowel syndrome (IBS), inflammatory bowel disease (IBD), Crohn's disease, fatty liver disease, fibrosis, drug-induced liver disease; neurological disorders such as Alzheimer's disease, multiple sclerosis (MS), Parkinson's disease, bovine spongiform encephalopathy (BSE, mad cow disease); cancers such as breast, renal, stomach, melanoma, lung, colon, glioma, lymphoma and prostate cancer.

In one embodiment the B lymphocytes are screened for the presence of antibodies against therapeutically relevant targets such as polypeptides associated with neuronal conditions, cytokines, chemokines, growth factors, adhesion molecules, co-stimulatory molecules, tumor cell antigens, malignant cell antigens and their receptors.

Polypeptides associated with various neurodegenerative diseases, such as Huntington's Disease (HD), Parkinson's Disease (PD), Alzheimer's Disease (AD), and Amyotrophic Lateral Sclerosis (ALS) include huntingtin, atrophin-1, androgen receptor, ataxin-1, ataxin-2, ataxin-3, CACNA1A (calcium channel, voltage-dependent, P/Q type, alpha 1A subunit), ataxin-7, α-synuclein, amyloid precursor protein (APP), tau, β-amyloid peptide, low-molecular-weight neuronal filament (LNF), α-internexin, peripherin, N-Cor, mSin3a, CBP (c-AMP-responsive-element-binding protein), α-adaptin, α-1-antichymotrypsin, synphilin-1, parkin, UCH-L1 (ubiquitin carboxyl-terminal esterase L1), hip-1, caspase-1, caspase-2, caspase-3, caspase-6, caspase-8, calpain, aspartyl protease, histone deacetylase 2 (HDAC2), transglutaminases, polyglutamine-binding-protein-1 (PQBP1), β-synuclein, γ-synuclein, SOD1, apolipoprotein E (APOE), hip-1, presenilin PS-1, and presenilin PS-2.

Cytokines are a heterogeneous group of polypeptide mediators that have been associated with activation of numerous functions, including the immune system and inflammatory responses. The cytokine families include, but are not limited to, interleukins (IL-1 alpha, IL-1 beta, ILIra and IL-2 to IL-18), tumor necrosis factors (TNF-alpha and TNF-beta), interferons (INF-alpha, beta and gamma), colony stimulating factors (G-CSF, M-CSF, GM-CSF, IL-3 and some of the other ILs), and growth factors (EGF, FGF, PDGF, TGF alpha, TGF betas, BMPs, GDFs, CTGF, and ECGF). Cytokines include but are not limited to cardiotrophin-1 (CT-1); CD27; CD27L; CD30 Ki-1; CD30L; CD40L (TRAP); interferon alpha (IFN-alpha); interferon beta (IFN-beta); interferon gamma (IFN-gamma); interferon omega (IFN-omega); interferon-sensitive gene 15 (ISG-15); Leptin OB; leukemia inhibitory factor LIF; Lymphotoxin LT/TNF beta; macrophage colony stimulating factor (M-CSF); macrophage stimulating protein-alpha (MSP-alpha); macrophage stimulating protein-beta (MSP-beta); migration inhibition factor (MIF); oncostatin M (OSM); RANKL; soluble IL6 R complex sIL6RC (gp130+sIL6R); soluble Fas ligand sCD95L; TNF type I receptor TNF-RI; TNF type II receptor TNF-RII; TNFSF-18; tumor necrosis factor alpha TNF-alpha; and TNFSF-12.

Chemokines are those cytokines that may activate or chemoattract leukocytes. Chemokine receptors belong to the G-protein-coupled class of receptors. For example, entry of HIV into host cells requires chemokine receptors, and their antagonists are useful for treatment of AIDS. Chemokines include but are not limited to B-lymphocyte chemoattractant (BLC); chemokine receptor (CCK-1); cutaneous T cell attracting chemokine CTACK; Eotaxin-1; Eotaxin-2 MPIF-2; Eotaxin-3 CCL26; neurotactin; Granulocyte chemotactic protein 2 (GCP-2); MGSA; MIP-2alpha; MIP-2beta; haemoinfiltrate CC 1 (HCC-1); haemoinfiltrate CC 4 (HCC-4); IFNgamma inducible protein-10 (IP-10); IFN-inducible T cell alpha chemokine (1-TAC); interleukin-8 (IL-8); leucocyte cell-derived chemotaxin-2; Lungkine; Lymphotactin (LPTN); macrophage inflammatory protein 1alpha; macrophage inflammatory protein 1beta; macrophage inflammatory protein 1 delta; macrophage inflammatory protein 1 gamma; macrophage inflammatory protein 3alpha; macrophage inflammatory protein 3beta; macrophage-derived chemokine (MDC); monocyte chemoattractant protein-1 (MCP-1); monocyte chemoattractant protein-2 (MCP-2); monocyte chemoattractant protein-3 (MCP-3); monocyte chemoattractant protein-4 (MCP-4); monocyte chemoattractant protein-5 (MCP-5); monokine induced by IFN gamma (MIG); Myeloid progenitor inhibitory factor (MPIF); platelet basic protein (PBP); platelet factor 4; pulmonary activation regulated chemokine (PARC); RANTES (regulated upon activation T cell expressed and secreted); secondary lymphoid tissue chemokine (SLC); stromal cell derived factor 1 (SDF-1); thymus activation regulated (TARC); and thymus expressed chemokine (TECK).

Growth factors include but are not limited to acidic fibroblast growth factor (aFGF); activin beta A; agouti related protein (AGRP); Amphiregulin AR; angiopoietin-like factor (ALF); basic fibroblast growth factor (bFGF); Betacellulin; bone morphogenic protein 2 (BMP2); bone morphogenic protein 4 (BMP4); bone morphogenic protein 5 (BMP5); bone morphogenic protein 6 (BMP6); bone morphogenic protein 7 (BMP7); cripto-1 growth factor (CRGF); epidermal growth factor (EGF); Erythropoietin (EPO); fibroblast growth factor 17 (FGF-17); fibroblast growth factor 18 (FGF-18); fibroblast growth factor 19 (FGF-19); fibroblast growth factor 2 (FGF-2); fibroblast growth factor 4 (FGF-4); fibroblast growth factor 6 (FGF-6); fibroblast growth factor 7 (FGF-7); fibroblast growth factor 8 (FGF-8); fibroblast growth factor 9 (FGF-9); Flt3 ligand (Flt3 L); Follistatin (FSP); Granulocyte colony stimulating factor (G-CSF); granulocyte/macrophage CSF (GM-CSF); growth and differentiation factor 11 (GDF-11); growth and differentiation factor 15 (GDF-15); growth arrest specific gene 6 (Gas-6); heparin-binding epidermal growth factor (HB-EGF); hepatocyte growth factor (HGF); hepatopoietin A (HPTA); neuregulin; heregulin alpha; heregulin beta; IGF binding protein-1 (IGFBP-1); IGF binding protein-2 (IGFBP-2); IGF binding protein-3 (IGFBP-3); IGF binding protein-4 (IGFBP-4); inhibin A; inhibin B; insulin-like growth factor IA (IGF-IA); insulin-like growth factor IB (IGF-IB); insulin-like growth factor II (IGF-II); macrophage galatose-specific lectin 1 (MAC-1); Neuritin; Neurturin; orexin A; Osteonectin; Osteoprotegrin; platelet derived growth factor alpha (PDGF-A); platelet derived growth factor beta (PDGF-B); prolactin (PRL); sensory and motor neuron-derived factor (SMDF); soluble GM-CSF receptor (sGM-CSF R); stem cell factor (SCF); Thrombopoietin (TPO); thymic stromal lymphoprotein (TSLP); Thymopoietin (Tpo); transforming growth factor alpha (TGF-alpha); transforming growth factor beta 1 (TGF-beta1); transforming growth factor beta 2 (TGF-beta2); transforming growth factor beta 3 (TGF-beta3); and vascular endothelial growth factor (VEGF).

Targeting cellular adhesion molecules and chemokines/chemokine receptors as regulators of the extravasation and migration of leukocytes provide an approach for the treatment of chronic inflammatory disorders such as rheumatoid arthritis and osteoarthritis. Vergunst C E et al., Scandinavian Journal of Rheumatology 34:6, 415-425. Cell Adhesion Molecules (CAMs) are proteins located on the cell surface involved with the binding with other cells or with the extracellular matrix (ECM) in the process called cell adhesion. Most of the CAMs belong to 4 protein families: Ig (immunoglobulin) superfamily (IgSF CAMs), the integrins, the cadherins and the selectins. Immunoglobulin superfamily CAMs (IgSF CAMs) are either homophilic or heterophilic and bind integrins or different IgSF CAMs. IgSF CAMs include but are not limited to: NCAMs (Neural Cell Adhesion Molecules); ICAM-1 (Intercellular Cell Adhesion Molecule); VCAM-1 (Vascular Cell Adhesion Molecule); PECAM-1 (Platelet-endothelial Cell Adhesion Molecule); L1; CHL1; MAG; Nectins and nectin-like molecules. Members of the cadherin family include E-cadherins (epithelial), P-cadherins (placental) and N-cadherins (neural). Examples of selectin family members are E-selectin (endothelial), L-selectin (leukocyte) and P-selectin (platelet). Integrins are cell surface receptors that interact with the extracellular matrix (ECM) and mediate various intracellular signals. Cell adhesion is implicated in infectious diseases and neurological disorders.

The co-stimulatory signal is an antigen nonspecific signal used during T cell activation and is provided by the interaction between co-stimulatory molecules expressed on the membrane of antigen presenting cell and the T cell. (Tacke et al., Eur. J. Immunol., 1997, 27:239-247.) An example of a costimulatory molecules expressed by T cells is CD28, which interacts with CD80 and CD86 on the membrane of APC. Other costimulatory receptors expressed by T cells include ICOS (Inducible Costimulator), CTLA-4, and PD1. Inhibition of costimulatory signal is used for treatment of rheumatoid arthritis and during renal transplantation as well as for the treatment of diseases lacking costimulability of T cells, in particular of chronic lymphocytic leukemia of the B-cell type (B-CLL), agammaglobulinemia, selective immunoglobulin deficiencies, such as selective IgA deficiency, and common variable immunodeficiencies (CVID).

Samples containing lymphocytes can be collected from the patient donor at various time points. In one embodiment, lymphocytes are collected from a patient who has recovered from the targeted disease(s) at least for 1, 5, 10, 15, 20, 25 days, at least for 1, 2, 3, 4, 5, 6, 7, 8, 9, 10, 11 months, or at least for 1, 2, 3, 4, 5, 6, 7, 8, 9, 10 years. In another embodiment, lymphocytes are collected from a patient who is having the targeted disease(s) at the time of collection, and has been diagnosed as having the disease(s) at least 1, 5, 10, 15, 20, 25 days, or at least 1, 2, 3, 4, 5, 6, 8, 9, 10 months, or 1, 2, 3, 4, or 5 years prior to collection.

In order to prepare donor-specific human antibody libraries, samples containing B-lymphocytes are collected from individuals (patient donors). The sample may, for example, derive from bone marrow, blood, spleen, lymph nodes, tonsils, thymus, and the like. While peripheral blood mononuclear cells are the most common source for samples, it is noted that bone marrow represents the complete “fossil archive” of individual donor's mature antibody repertoire, and mononuclear cells in the spleen contain a higher percentage of IgG antibodies. The best sources of primary human B cells are splenic mononuclear cells, tonsils and peripheral blood mononuclear cells. (Olsson et al. J. Immunol. Methods 61:17-32 (1983); Karpas A. Proc. Natl. Acad. Sci. USA 98:1799-1804 (2001)).

The procedure begins with separation of peripheral blood mononuclear cells (PBMC) from human blood as known in the art, and typically by use of a Ficoll gradient. The PBMC are stained with the B cell selective marker, such as anti-CD19. Stained B cells are sorted by flow cytometry. In one aspect of the invention, about 5-10×10⁴ B-cells are obtained per 5 mL blood sample.

### Cloning Antibody-Producing B-Cells

Antibody-producing B-cells may be cultured in multi-well plates. In one embodiment, each well of a 96, 384 or 1536 well plate is oligoclonal and contains more than one B cell clone. A well may contain at least 1, 2, 5, 10, 15 or 20 different B-cell clones, preferably between 1-100 B cell clones. Preferably a well contains about 10 different B-cell clones. High density libraries may be constructed according to Love et al., Nature Biotechnology, 24, pp. 703-707 (2006) (“Love”). Preferably the B cells are disposed within microtiter plates; more specifically 96, 384 or 1536 well microtiter plates. The benefit of using a microtiter plate format (e.g., compared to the nano-format of Love) is ease of retrieval of the B cells. It is contemplated that the microtiter plates may comprise multiple B cells in a single well, with a plurality of the B cells in each well producing different human native antibodies. In another embodiment, each well of a 96, 384 or 1536 well plate is clonal and contains on average no more than one B cell clone; this embodiment is preferred when the human B cells are not immortalized.

Two alternatives for sorting cells into microtiter plates at limiting dilutions of about 10 cells per well include picking clones from semi-solid medium (Davis, J. M., et al. J. Immunol. Methods 50, 161-171 (1982); Rueda, A. Z. & Coll, J. M. J. Immunol. Methods 114, 213-217 (1988)) and fluorescence-activated cell sorting (FACS; Herzenberg, L. A. et al. Clin. Chem. 48, 1819-1827 (2002); Carroll, S. & Al-Rubeai, M. Expert Opin. Biol. Ther. 4, 1821-1829 (2004)).

Optionally, the B-cell clones are expanded in the wells. Stimulation of B-cells in vitro results in production of more immunoglobulin mRNA per cell, division of the cell leading to clonal expansion and enhanced production of soluble immunoglobulin which is released into the medium.

Various methods for effective in vitro stimulation of the primary B-cells have been described. Zubler and co-workers (Wen et al., Eur J. Immunol. 1987 17: 887) described the use of mutant EL4 subclone, EL4-B5 as stimulator/feeder cells in B-cell cultures. Banchereau and co-workers (Valle et al., Eur J. Immunol. 1989 19: 1463) described the use of agonistic anti-CD40 monoclonals, displayed on Fc-gamma receptor expressing fibroblasts used as feeder cells. More recently, CD40L transfected cell lines have been used as stimulator/feeder cells (Armitage et al., Nature. 1992 357: 80 and Spriggs et al., J Exp Med. 1992 176: 1543) as well as recombinant soluble fragments of CD40L (Hollenbaugh et al., EMBO J. 1992 11: 4313 and Mazzei et al., J. Biol. Chem. 1995 270: 7025). U.S. Pat. No. 5,540,926 describes a method for promoting B-cell proliferation comprising exposing activated B-cells in vitro to an effective concentration of a soluble gp39 protein. Treatment of primary B cells with a proliferative stimulus prior to hybridoma fusion with pokeweed mitogen or EBV has been described. (Olsson et al. J. Immunol. Methods 61:17-32 (1983); Butler J L et al. J. Immunol. 130:165-168 (1983)). U.S. Pat. No. 5,851,531 describes a method for B cell stimulation by pokeweed mitogens comprising lectins from the pokeweed plant, Phytolacca americana. It is known that immune stimulatory effects of oligodeoxynucleotides containing unmethylated CpG dinucleotides in particular base contexts (CpG motifs) have highly stimulatory effects on human leukocytes, inducing B cell proliferation. (Krieg, 1999 Biochim. Biophys. Acta 93321:1-10; Krieg, A. M., Applied Antisense Oligonucleotide Technology, 24:431-448 (1998)).

Release of soluble immunoglobulin into the medium by stimulated B-cells enables one to conveniently screen B-cell cultures for the presence or absence of antigen-specific heavy-chain antibodies. For instance, one can test the conditioned supernatant by removing the conditioned medium from the cells and use all or part of the sample in an immunoassay configured to quantify immunoglobulin concentrations present in the medium to reveal which stimulated cultures contain successfully stimulated B-cells. This enables one to exclude unsuccessfully stimulated B-cell cultures in subsequent steps of the immunoglobulin gene cloning procedure.

### Immortalization of B Cell Clones

Primary human B cells producing native human antibodies are immortalized in situ by EBV transformation, hybridoma formation, or a combination thereof, and banked. Hybridoma methods for cloning these antibodies have many potential advantages, including convenience, high-yield antibody expression, and the ability to capture the antibodies in their native configurations.

B cell clones can be expanded by techniques known in the art including the use of hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981)

In an improved hybridoma production method, Dessain et al. (J. Immunol. Methods 291, 109 (2004)) demonstrated that stable human B-cell hybridomas can be created using a murine fusion partner cell line (MPT) that expresses human telomerase (hTERT) and murine interleukin-6 (mIL-6).

Another well known method for expanding human B cell lines is transformation using Epstein Barr Virus (EBV). Protocols for generating EBV-transformed B cell lines are commonly known in the art, such as, for example, the protocol outlined in Chapter 7.22 of Current Protocols in Immunology, Coligan et al., Eds., 1994, John Wiley & Sons, N.Y., which is hereby incorporated by reference in its entirety. Tissues are generally made into single cell suspensions prior to EBV transformation. Additionally, steps may be taken to either physically remove or inactivate T cells (e.g., by treatment with cyclosporin A) in B cell-containing samples, because T cells from individuals seropositive for anti-EBV antibodies can suppress B cell immortalization by EBV.

In general, the sample containing human B cells is inoculated with EBV, and cultured for 3-4 weeks. A typical source of EBV is the culture supernatant of the B95-8 cell line (ATCC #VR-1492). Physical signs of EBV transformation can generally be seen towards the end of the 3-4 week culture period. By phase-contrast microscopy, transformed cells may appear large, clear, hairy and tend to aggregate in tight clusters of cells. Initially, EBV lines are generally polyclonal. However, over prolonged periods of cell cultures, EBV lines may become monoclonal or polyclonal as a result of the selective outgrowth of particular B cell clones. Alternatively, polyclonal EBV transformed lines may be subcloned (e.g., by limiting dilution culture) or fused with a suitable fusion partner and plated at limiting dilution to obtain monoclonal B cell lines. Suitable fusion partners for EBV transformed cell lines include mouse myeloma cell lines (e.g., SP2/0, X63-Ag8.653), heteromyeloma cell lines (human×mouse; e.g., SPAM-8, SBC-H20, and CB-F7), and human cell lines (e.g., GM 1500, SKO-007, RPMI 8226, and KR-4).

In a recently improved EBV-immortalization method, human primary CD19⁺IgG⁺B cells are stimulated with a CpG Oligonucleotide prior to EBV exposure. (Hartmann and Krieg. J. Immunol. 164:944-953 (2000)).

This procedure results in a library of clonally expanded IgG⁺ memory B cell cultures each capable of producing 1, 2, 3, 4, 5 and/or 10 distinct IgG species. These hybridomas or EBV-immortalized cells can be stored as sources for the specific antibody species represented in each well.

### Non-Immortalized B-Cell Libraries as Sources for Antibodies

The conditioned supernatant from the corresponding B-cells prior to supernatant analysis are separated and all B-cell cultures during analysis of the supernatants are saved. B-cells present in the original B-cell culture of the wells corresponding to antibodies of the most interest can be retrieved and used to rescue the native human IgG-encoding mRNA using methods known in the art. In one aspect of the invention a library of such B-cells is generated, each corresponding to a specific antigen specificity and/or each representing 1, 2, 5, 10 or 20 native human IgG producing B-cell clones and banked and stored (for example, as frozen pellets).

B-cell pellets from which the conditioned supernatant has been removed for analysis can be stored in various ways during conditioned supernatant analysis: as intact frozen cells using media suitable for storing live mammalian cells (i.e. cell culture medium containing 10% DMSO), as frozen cell lysates prepared by lysing the cell pellets using an RNA protective cell lysis solution (i.e. TRIzol®, Invitrogen (Carlsbad, Calif.)) or in a buffer designed to protect RNA from degradation at room temperature or below without lysing the cells (i.e. RNAlater®, Ambion (Austin, Tex.)).

Strategies for cloning and expressing antibodies from single human B cells of defined origin are known in the art (Wardemann et al., Science 301:1374-1377 (2003)). According to a subsequent aspect of the invention, RNA is isolated from the stored B-lymphocytes. The RNA obtained is a collection of nucleic acids, already selected from the immune repertoire, and contains mRNAs encoding native human immunoglobulins. In one aspect of the invention, the immunoglobulins are pre-selected for binding antigens of interest. Methods to isolate RNA are known in the art (Liedtke et al. PCR Methods Appl. 1994 December; 4(3):185-187) and include TRIzol® reagent (Invitrogen). Sufficient quantities of RNA can be obtained from non-immortalized antigen-specific B-lymphocytes for the rescue of antibodies by RT-PCR.

Using species-specific oligonucleotides which hybridize to sequences flanking nucleic acid sequences encoding the antibody genes, methods such as single-cell reverse transcriptase PCR are used to amplify variable heavy and light chain nucleic acid sequences or fragments thereof. (Coronella, et al. (2000) Nucleic Acids Res. 28(20):E85) For example, human variable heavy and light chain antibody domains can be PCR-amplified using human-specific oligonucleotides (see, e.g., Sblattero and Bradbury Immunotechnology 3:271-278 (1998)). Amplified sequences can be characterized by DNA sequencing and directly cloned as individual sequences into an expression system. Other techniques for amplifying immunoglobulin sequences of conventional 4-chain antibodies from individual B-cells are described in Takahashi et al., Journal of Biotechnology 49 (1996), 201-210; and Embleton et al., Nucleic Acids Research, Vol. 20, No. 15, 3831-3837. Methods using nested RT-PCR for amplifying heavy and corresponding light chain gene transcripts from single human B cell clones isolated by fluorescence-activated cell sorting are described by Tiller et al. (J Immunol Methods. 329(1-2):112-124 (2008)).

Subsequently, the amplified nucleic acid sequences can be introduced into a suitable expression system for storage and future use. Methods for producing recombinant proteins such as antibodies in expression systems are well-known in the art. In general, nucleic acid sequences encoding the antibody are incorporated into a recombinant expression vector in a form suitable for expression of the antibody, or fragment thereof, in a host cell. A suitable form for expression provides that the recombinant expression vector includes one or more regulatory sequences operatively-linked to the nucleic acids encoding the antibody, or fragment thereof, in a manner which allows for transcription of the nucleic acids into mRNA and translation of the mRNA into the protein. Regulatory sequences may include promoters, enhancers and other expression control elements (e.g., polyadenylation signals) and are known to those skilled in the art (Goeddel D. D., ed., Gene Expression Technology, Academic Press, San Diego, Calif. (1991)). It should be understood that the design of the expression vector may depend on such factors as the choice of the host cell to be transfected and/or the level of expression required.

In one embodiment, for reverse-transcriptase-polymerase chain reaction (RT-PCR) rescue of the immunoglobulins, a freshly prepared master mix of primers and enzyme/nucleotide mix for both RT and PCR reactions are added to all wells of freshly thawed PCR strips containing sorted B-cells which have been stored at −80° C. Both reverse transcription reaction and PCR amplification of the cDNA are performed sequentially in the same tube, using the same or different suitable 3′ primers for both reactions. Reactions are run on a thermocycler under conditions known in the art. Once both RT and PCR reactions have been run, the reaction mixture is analyzed (for example, on a SYBR® Safe stained agarose gel). PCR reactions found to contain an amplicon are purified. For example, using Qiagen PCR purification spin columns (Qiagen), amplicons are purified and digested using suitable restriction enzymes and the digests are purified via agarose gel using Qiaquick gel extraction kit (Qiagen). The DNAs corresponding to light and heavy chains of the native human immunoglobulins then are ligated into pre-digested expression vectors containing inducible promoters and periplasmic space export leader signals using standard methods. Ligation mixtures were introduced into competent cells through electroporation and grown on selective medium. Individual colonies are screened for the presence of plasmid insert using colony PCR with two primers annealing to sequences located shortly 5′ and 3′ of the cloning site, respectively, and detection of PCR amplicon length using SYBR Safe stained agarose gels. Cloning of the light and heavy chain genes can be confirmed by sequencing. We contemplate a method of making a library using the steps of obtaining at least 10⁴ memory B-cells from each of an effective number of human donors, preparing a population of human B-cells, wherein said population contains at least 10⁵ different species of naturally occurring antibodies wherein each of the antibodies has naturally paired heavy and light chains, dividing said population of B cells into subpopulations of B cells each subpopulation produces on average 1-100 different species of antibodies, optionally, expanding each subpopulation of B cells to produce an expanded B-cell culture; and storing each sub-population under conditions suitable for preserving its RNA content, wherein a library of non-immortalized B-cell populations each expressing on average 1-100 different species of antibodies is produced. In further steps we contemplate preparing RNA samples corresponding to a plurality of the stored sub-population of B-cells, performing reverse transcriptase-polymerase chain reaction (RT-PCR) on a plurality of the RNA samples, and isolating DNA corresponding to V_{H} and V_{L} regions capable of natural pairing. In still further steps we contemplate cloning said DNA corresponding to V_{H} and V_{L} regions in a suitable host capable of expression of said V_{H} and V_{L} regions and expressing said V_{H} and V_{L} regions in the context of an immunoglobulin heavy and light chain, such that a naturally paired immunoglobulin is formed.

Screening of antigen reactivity of the cloned native human IgG genes can be performed on replicates of the same cultures used for sequencing. Extracts of the cultures can be screened for binding in parallel on antigen coated ELISA plates.

### Screening B-Cell Libraries for Antibodies Against Specific Antigens

Using the B-cell conditioned supernatants in immunoassays configured to detect antigen binding immunoglobulin allows one to determine which wells contain stimulated B-cells encoding immunoglobulins binding the antigen. The reagents required for immunoglobulin selective immunoassays are available to one of skill in the art. For example, such reagents may include, but are not limited to, polyclonal or monoclonal antibodies generated against light and/or heavy chains of antibodies. Methods to prepare and characterize such polyclonal or monoclonal antisera are well known to those skilled in the art. Non-limiting examples of reagents suitable for detection of such markers as described above are given in Daley et al. (Clin Diag Lab Immunol. 2005 12: 380).

Release of soluble immunoglobulin into the medium by stimulated B-cells enables one to conveniently screen B-cell cultures for the presence or absence of antigen-specific heavy-chain antibodies. For instance, one can test the conditioned supernatant by removing the conditioned medium from the cells and use all or part of the sample in an immunoassay configured to quantify immunoglobulin concentrations present in the medium to reveal which stimulated cultures contain successfully stimulated B-cells. This enables one to exclude unsuccessfully stimulated B-cell cultures in subsequent steps of the immunoglobulin gene cloning procedure. Use of such a screening assay allows one to focus the downstream cloning of immunoglobulin genes towards the only relevant B-cell clones (antigen specific, native human immunoglobulin producing cells).

Having access to stimulated B-cell conditioned supernatants also enables one to screen for B-cell clones producing immunoglobulin having desirable functional characteristics, such as being able to neutralize receptor/ligand interaction where either one is the antigen in question, having an agonistic or antagonistic effect on receptor activation, having high antigen binding affinity or being able to inhibit enzymatic activity. Screening for such characteristics can be performed on antibody isolated from conditioned supernatants collected off the B-cell cultures, but usually can be performed more conveniently on the conditioned supernatant itself. Methods for screening antibody containing solutions such as B-cell conditioned supernatants for the type of activities mentioned above are known to those skilled in the art. Both heterogeneous methods (such as chromogenic, fluorescent or radioactive readout immunoassays in plates, on beads or microarrays and bioassays) as well as homogeneous assays (such as LANCE®, Alphascreen® or using confocal imaging systems such as ABI's FMAT® or Evotech's Opera®) are suitable for binding and activity assays. As methods for affinity determination, bioassays, surface plasmon resonance or cantilever MEMS based devices as well as off-rate selective immunoassays (Friguet et al., J Immunol Methods. 1985 77: 305) are mentioned as non-exclusive examples.

In one embodiment, the antibody producing B-cells are screened prior to clonal expansion. Love et al (Nature Biotech. 24(6): 703-707 (2006) describe a soft lithographic technique for microengraving that uses a dense array of microwells (0.1-1 nl each) containing individual cells to print a corresponding array of molecules secreted by each cell. The cells remain in culture after engraving, and the microarrays are interrogated in a manner similar to commercial microarrays of proteins or antibodies. This method enables rapid identification of those cells exhibiting desired properties, such as secretion of an antigen-specific antibody, and their subsequent recovery for clonal expansion.

The antibodies produced by the B cell culture supernatants may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety).

### Generating Antibody Repertoire Arrays (ARAs)

Materials for ARAs are generated by culturing immortalized clones for the production of secreted IgG antibodies. Human immunoglobulin secretion can be analyzed using standard techniques for ELISA assays (E. Harlow, D. Lane, Antibodies: A laboratory manual. (Cold Spring Harbor Laboratory, Cold Spring Harbor, 1988)). In one example, wells in standard 96-well or 384-well ELISA plates are coated with primary rabbit anti-human IgG specific for heavy and light chains antibodies. The same antibody, conjugated to horseradish peroxidase, is used as a secondary at a 1:3000 dilution in phosphate buffered saline/0.1% bovine serum albumin. Assays are developed using standard techniques with a chromogenic substrate.

B-cell culture supernatants expressed from the expanded B-cell culture in each well is used to create the Antibody Repertoire Array (ARA). Typically, 10⁴ to 10⁵ features, preferably 5×10⁴ features are printed in duplicate on each ARA. Several techniques for such printing are known in the art.

The formation of an ARA representative of the human immunome requires the immobilization of antibodies on a solid substrate without loss of activity for ELISA microarrays. Proteins are structurally more complex molecules than DNA, and can unfold and lose activity when immobilized on a solid substrate due to hydrophobic or ionic interactions with the surface. There is also potential for proteins to denature during the drying process. The capture antibody for a microarray ELISA is printed at low volume (0.3 to 1 nL). The capture antibody spots dry quite rapidly due to the low print volume, and long-term storage conditions typically require the chip to be dry. While antibodies are more stable than most proteins, there is still potential for a loss of activity upon drying and storage.

There are three general categories of immobilization chemistries whereby antibodies are attached to glass slides: (i) physical adsorption, (ii) covalent attachment via reactive groups, and (iii) affinity-based interactions between functional groups on the slide and the antibody. (Reviewed in Seurynck-Servoss S L et al., Frontiers in Bioscience 12:3956-3964 (2007).

(i) Physical adsorption of proteins occurs via hydrophobic or ionic interactions between the protein and a slide surface coated with, for example, agarose, polyacrylamide, nitrocellulose, poly-L-lysine, or aminosilane. While this is a simple immobilization technique, it is not easily controlled and may result in high variability as well as undesirable random orientation of antibody molecules on the surface. Antibodies randomly immobilized on the surface may cause the antigen binding regions of some antibodies to be directly attached to the glass surface and thus, inaccessible.

(ii) Covalent binding mediated by functional groups including primary amines in lysines or arginines, reactive thiols in the cysteines in the hinge region, or carbohydrates linked to the H2 domains of the constant (Fc) region can be used to permanently immobilize antibodies on a surface. Although attachment through thiols or carbohydrates allows for directed orientation of antibodies, the protocol for attachment is more complex. Specifically, the disulfide bonds must be reduced or the carbohydrate groups must be oxidized prior to attachment to the surfaces reactive towards these groups. These redox reactions can destabilize the antibody structure and decrease activity and may require additional purification steps.

The most commonly used surface chemistries for covalent immobilization of antibodies are epoxides, aldehydes, and N-hydroxy succinimidyl esters, all of which are reactive towards primary amines on the protein surface. Hydrazine coated surfaces attach through carbohydrate residues and maleimide coated surfaces attach through thiol residues.

(iii) The immobilization of antibodies through affinity-based interactions typically utilizes a unique functional group or protein sequence on the antibody, resulting in orientation of the antigen-binding sites. Some current techniques used for affinity-based immobilization of antibodies are (i) protein A or G coated slides, which have a high affinity for the Fc region of antibodies (Kusnezow, W. & J. D. Hoheisel: Journal of Molecular Recognition, 16, 165-176 (2003); Anderson, G. P., et al. Biosensors and Bioelectronics, 12, 329-336 (1997) or (ii) affinity slides that are specific for a unique tag in the antibody (Cha, T., et al. Proteomics, 5:416-419 (2005); Wingren, C., et al. Proteomics, 5:1281-1291 (2005)). Immobilization via an Fc specific antibody is attractive because commercially available monoclonal antibodies can be used without any further processing. Proteins A and G are specific for only certain IgG subclasses and can not be used universally with all monoclonal antibodies. The affinity of protein A or G varies with respect to antibody species as well as with buffer conditions. Therefore, it may not be possible to use protein A or G to immobilize all antibodies under all conditions and anti-human Fc antibodies may be used instead.

Streptavidin-biotin interaction has a very high affinity, and studies have shown that immobilization of antibodies via the streptavidin- or avidin-biotin interaction can result in highly sensitive assays (Delehanty, J. B. & F. S. Ligler. Analytical Chemistry, 74, 5681-5687 (2002)). However, it is necessary to use biotinylated antibodies for capture on streptavidin- or avidin-coated slides. The biotin can be chemically added. Arrays comprising antibodies spotted on poly-L-lysine coated glass with a cross-linking layer (Haab, B. B. et al. Genome Biol. 2, research 0004.1-0004.12 (2001)) and IgG arrays on poly-L-lysine (CEL Associates, Pearland, Tex.) with a photoreactive cross-linking layer (Molecular Biosciences, Boulder, Colo.) or polyacrylamide-based hydrogel (Packard Bioscience, Meriden, Conn.) glass slides have been described. (Miller, J C et al. Proteomics 3, 56-63 (2003)).

While a typical ARA comprises the B-cell culture supernatants expressed from the expanded B-cell culture in each well, it may also contain positive controls for the target antigen to be tested, as well barcodes and similar identifying information regarding the composition of the ARA.

In one embodiment, we contemplate printing or spotting the ARA with more than one unique antibody per spot or site on the ARA, preferably at least 1 antibody clone per spot, more preferably between 1-50 antibody clones per spot, even more preferably between 10-20 antibody clones per spot.

### Screening ARA for Target Binding

ARA can be screened by interrogation with native protein, peptides or other molecules representative of any antigen, including any agent or disease condition. Methods for screening are reviewed by Haab BB (Molecular & Cellular Proteomics 4:377-383 (2005)). Methods for using antibody arrays for high throughput screening and quantitative profiling of proteins are known in the art. (Chaga G S 441:129-151 in Tissue Proteomics, B. C.-S. Liu and J. R. Ehrlich eds., Methods in Molecular Biology (2008) Springer-Verlag (NY); Cahill D., Journal of Immunological Methods, 250(1-2): 81-91 (2001); Sanchez-Carbayo M., Clin Chem. 52(9):1651-1659 (2006)). Methods for use of antibody arrays in cancer proteomics is discussed in Sanchez-Carbayo M., Methods Mol Biol. 428:263-87 (2008); and Kopf et al., Int J Biochem Cell Biol. 39(7-8):1305-1317 (2007).

The ARA may comprise antibodies coating the surface of a microarray. The ARA may be interrogated with the antigen of interest conjugated to a detectable compound such as a fluorescent, chemiluminescent or bioluminescent tag or an enzymatic substrate (e.g., horseradish peroxidase or alkaline phosphatase) added to the ARA and incubated for a period of time, thereby detecting the presence of a suitable antibody. A second antibody conjugated to a detectable compound may be added following the addition of the antigen of interest to the coated well. Any suitable label or screening tool for detection may be used for interrogation. One of skill in the art would be knowledgeable as to the parameters that can be modified to increase the signal detected as well as other variations of ELISAs known in the art. For further discussion regarding ELISAs see, e.g., Ausubel et al, eds, (1994), Current Protocols in Molecular Biology, Vol. 1, John Wiley & Sons, Inc., New York, section 11.2.1.

In some embodiments the ARA can be screened directly with whole viruses or cells where the antigen is expressed on the cell surface. In this embodiment the virus or cell is immobilized on the ARA and is detected by any known detection technology, including those discussed above.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Rescue of Antibody-Producing Clones from Wells Corresponding to ARA “Hits”

Immortalized antibody-producing clones may then be deconvoluted by limit-dilution culture followed by detection of positive antibody by reverse capture ELISA. B-cell clones identified to produce native human antibodies against a specific antigen are serially diluted to a single B-cell per well concentration and screened for production of desired antibodies by reverse IgG capture ELISA. Thus individual antibody producing B-cell hybridomas that are specific for a specific antigen can be identified and isolated.

In one embodiment, the “rescue” of the desired native human antibodies involves expression of cloned heavy and light chains corresponding to their native pairings, in a suitable host. Coronella (Nucleic Acids Res. 28(20):E85 (2000)) discloses a method for amplification of human immunoglobulin heavy and light chains from single B lymphocytes isolated by FACS. Using a nested RT-PCR protocol, Coronella (2000) describes a method for recreating in vivo pairings of V_{H} and V_{L} regions from large numbers of cells. Tiller (J. Immunol Methods. 329(1-2):112-124 (2008)) also describes methods for amplifying heavy and corresponding light chain gene transcripts from single human B cell clones isolated by fluorescence-activated cell sorting using nested RT-PCR. Tiller (2008) further describes reversion of somatically mutated Ig genes to their germline sequences, cloning the immunoglobulin genes from single human B cells into eukaryotic expression vectors and producing recombinant antibodies in a human kidney cell line. The teachings of the methods of Coronella (2000) and Tiller (2008) are expressly incorporated herein by reference in their entirety. Recombinant native paired human antibodies can be screened by methods such as ELISA and immunofluorescence assays. By this method, cell lines expressing recombinant native paired human immunoglobulins are obtained. This invention relates to the recombinant human Ig and the cell lines that express the recombinant Ig wherein the recombinant Ig comprises native light and heavy chain pairings.

### Antibody Characterization and Lead Selection/Validation

Once a B-cell hybridoma or EBV-immortalized clone is identified as producing a desired species of IgG, the immortalized B-cell line can be produced in large scale by standard methods known in the art to produce milligrams quantities of the so-called “hit” antibodies. (See Monoclonal Antibody Production, The National Academies Press (1999)).

Such immortalized B-cell lines can be further characterized by rescue of the corresponding V_{H} and V_{L} genes from a single antibody-producing clone. Additional procedures such as cloning and sequencing of the V_{H} and V_{L} genes by methods known in the art can be used to propagate the novel immortalized B-cell lines.

### Discovery of New Functional Epitopes Using ARA Platform

The Antibody Repertoire Array (ARA) of the invention provides a high-throughput platform to facilitate identification of new functional epitopes and corresponding human monoclonal antibodies (mAbs) from B cells of protected subjects. The platform allows discovery of human Abs that bind a target in its native conformation. A library of antibodies can be generated wherein different epitopes of a native target or antigen are bound by different mAbs. Since different epitopes on a given protein or antigen can be related to different functional characteristics of the target protein, the ARA platform can provide identification of multiple functional epitopes targeted by human immune system. The ARA platform can typically be used to screen hundreds of human subjects who have been exposed to a particular disease-specific antigen. Since each subject provides in the order of about 10⁵ IgG species, the ARA platform is useful for generating a high-throughput libraries of mAbs targeted to almost all functional epitopes that can be targeted by the human immune system. The high throughput process enabled by the ARA platform allows rapid screening based on samples from hundreds of donors in a miniaturized, microarray-based screening format with low reagent usage.

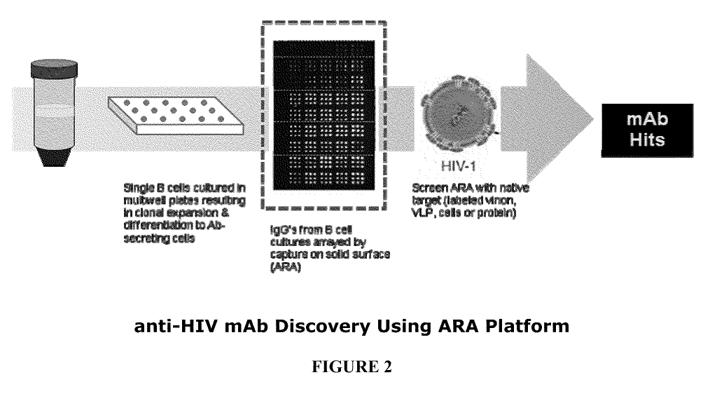

In one aspect of the invention, the ARA platform is used to recover ˜10⁷ recombinant IgG species directed against a viral target such as the human immunodeficiency virus (HIV), as shown in FIG. 2. In this example, blood samples from subjects exposed to HIV and containing IgG⁺ memory B cells are provided. Single B cells are cultured in multiwell plates resulting in clonal expansion and differentiation to antibody-secreting cells. An ARA is formed by immobilization of IgGs from the individual B cell culture. The ARA is then screened with native viral targets corresponding to HIV infection. Such targets may be selected from whole virion or virus-like proteins, individual proteins (for example, surface or envelope protein), or cells infected with HIV. B cell cultures corresponding to target-binding spots of the ARA are identified and recombinant IgG is rescued from lysed B cells isolated from each culture.

This method for anti-HIV mAb discovery using the ARA platform provides an archive generated from human IgG⁺ memory B cells which correspond to potentially protective anti-viral responses. The screening is performed with targets in native conformation thus producing more relevant results.

In one aspect of the invention, parallel screening of multiple targets from within a given strain or the same target protein derived from diverse strains of HIV, is performed. This results in identification of antibodies with broad cross-reactivity.

### Discovery of Antibodies Corresponding to Individual Epitope Clusters on a Given Target

The erbB2 oncogene encodes a growth factor receptor whose overexpression correlates with more aggressive tumors and a poorer prognosis. Some antibodies directed to this molecule have an antitumor effect in vivo, but some antibodies do not. Analysis of binding epitopes on erbB2 for inhibitory (HERCEPTIN®) and non-inhibitory (HF) antibodies by computer-guided protein engineering and site-directed mutagenesis revealed two different binding interactions. (Wang et al. Mol Immunol. (2004) 40(13):963-969). Non-inhibitory antibody HF only recognized N-terminal portion of erbB2 ectodomain (ECD), whereas the inhibitory antibody HERCEPTIN® bound to C-terminal portion of it exclusively.

The ARA screening platform can be used for identification and characterization of antibodies directed against different epitopes on a given target. This enables discovery on antibodies with potential activity against specific functions associated with each epitope cluster.

In this aspect of the invention, a phage display library, members of which express parts of a protein derived from gene fragments of a given target is used to identify the epitope repertoire for the target for a given immune response against that target. In one embodiment, functional assays of the target in conjunction with standard techniques, such as site-directed mutagenesis, is used correlate individual or groups of gene fragments with specific functions.

The ARA screening platform is used to cluster anti-target Abs by epitope specificity as determined by gene fragments provided by the phage display. Detailed characterization (e.g., sequencing) of a sample of representative Abs from functional epitope clusters can then be used to reveal further characteristics of interactions that may be used to positively or negatively the functions associated with each cluster.

In one aspect of the invention, pair-wise analysis using two or more epitope clusters is used to identify potentially cryptic functional epitopes. Such cryptic epitopes may act synergistically to enhance the function associated with a different epitope associated with a known function. The method allows for expanded exploration of the epitope space as compared to what is available in the literature based on pre-existing methods.

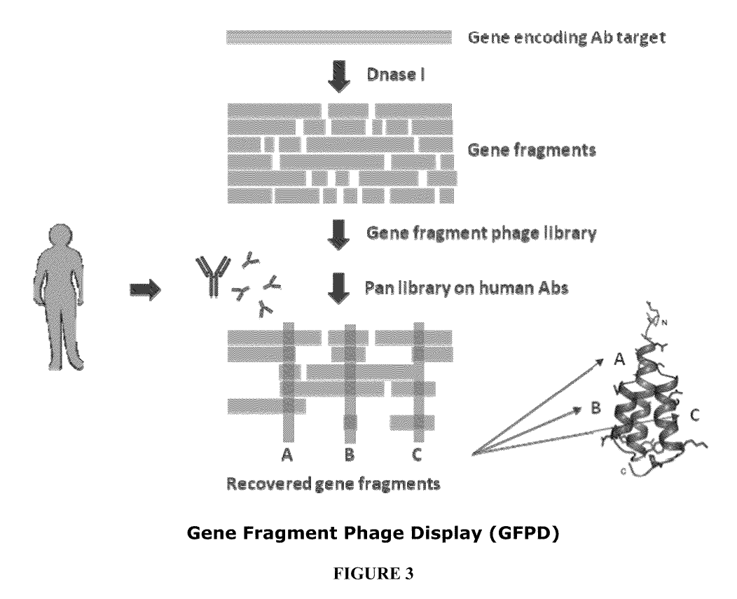

In one embodiment a gene fragment phage display (GFPD) library is generated as shown in FIG. 3. A gene fragment phage display expression library can be generated by methods known in the art. (See Silverman G. J., Chapter 20: Construction and Selection from Gene Fragment Phage-Display Expression Libraries, in Phage Display: A Laboratory Manual by Carlos F. Barbas III, Dennis R. Burton, Jamie K. Scott, Gregg J. Silverman, © CSHL Press, 2004). Gene fragments are generated by digestion of the gene encoding a given target by digestion with an endonuclease. A GFPD library is then generated by inserting the gene fragments into the genomic DNA of a phage in a way that parts of the target protein will be expressed on the surface of the phage. By “panning” the GFPD library on human antibodies directed against the target, a human epitope repertoire comprising recovered gene fragments is obtained. Gene fragments (GFPD library members) corresponding to different epitopes A, B, C, etc. are identified. In one embodiment, the gene fragments are overlaid on a known three-dimensional structure of the target protein to decide which gene fragments correspond to a particular epitope.

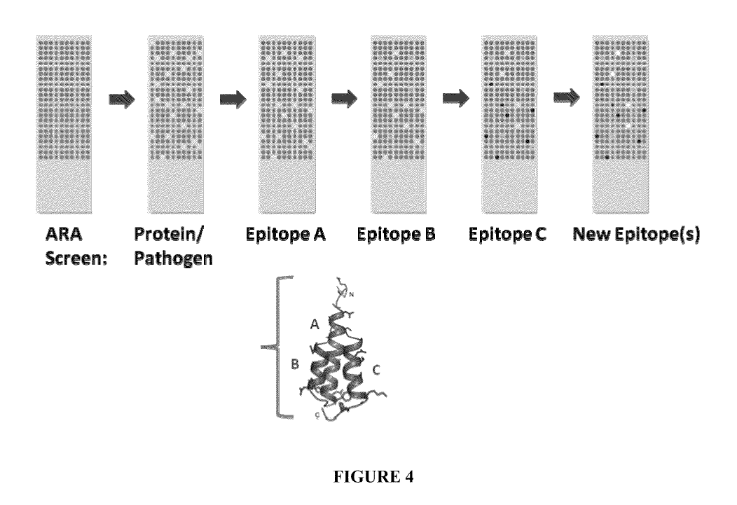

Screening on the ARA platform with the intact protein or pathogen target usually results in numerous hits, as shown in FIG. 4. Further screens of identical ARAs with GPDL members corresponding to epitopes A, B, C, etc are then performed. Typically, 2-3 GPDL members are screened per epitope, although there is no upper limit to the number of GPDL members per epitope that can be screened. Comparison of the “hit” pattern for the intact protein with those generated by the epitope-specific gene fragment clusters may also reveal novel epitopes that have not been identified before. Preferably, the epitope-specific gene fragments as well as the intact target both are recognized by the antibody. Thus, thousands of “hits” on a particular target antigen can be resolved into 10, 20, 30, 40 or more antibody “families” corresponding to epitope clusters. The antibodies are further characterized by sequencing the genes corresponding to V_{H} regions of antibodies that recognize the novel epitopes.

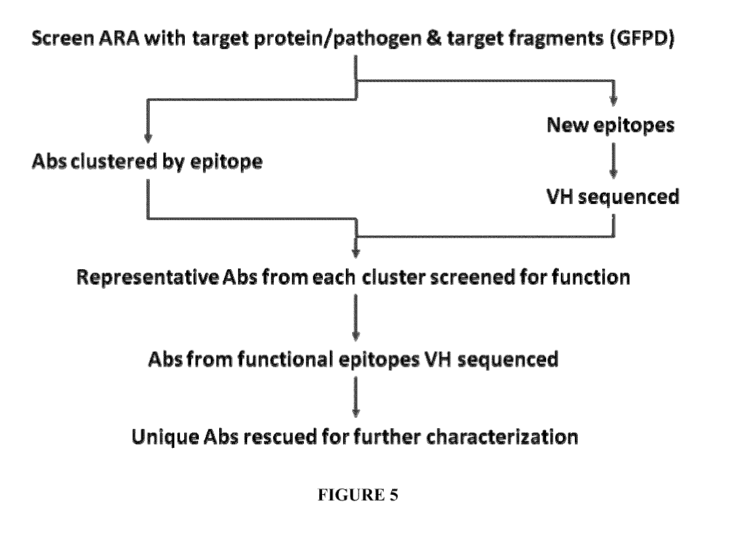

FIG. 5 shows a schematic diagram of the steps involved in using the ARA platform for identifying antibodies directed against functional epitopes. Representative antibodies against known and newly-identified epitopes that have been screened for functional correlation are sequenced at the V_{H} regions and rescued for further development. By this method unique antibodies can be identified that are suitable for development of therapeutics, and active and passive vaccines effective against specific functions associated with a target. The invention also relates to specific antibody libraries, antibodies, and therapeutics and vaccines effective against specific targets derived from the antibodies obtained by the methods of the invention.

### Function-Based Screening of Antibodies Using ARAs

Asthma is a complex inflammatory disease of the lung characterized by airway hyperresponsiveness (AHR), eosinophilic inflammation, mucus hypersecretion, subepithelial fibrosis, and elevated IgE levels. Interleukin-13 (IL-13) is a critical mediator of the effector phase of the allergic response in asthma. (Huang SK, et al. J. Immunol. (1995); 155(5):2688-2694). Anti-IL-13 antibodies that are useful in treating asthma generally block signaling pathways related to IL-13. (WO/2005/062967). IL-13 is also associated with Hodgkin's Disease (HD) and is found to be over-expressed in HD-derived cell lines. (Kapp, U., et al. J. Exp. Med., Volume 189, Number 12, 1999; 1939-1946). Anti-IL-13 antibodies that are useful in Hodgkin's Disease affect receptor binding by IL-13.

Monoclonal antibody (MAb) 263 is a widely used monoclonal antibody that recognizes the extracellular domain (ECD) of the Growth Hormone (GH) receptor and shown to act as a GH agonist both in vitro and in vivo. (Wan Y., et al., Molecular Endocrinology 17 (11): 2240-2250 (2003)). A murine MAb, termed BAH-1, raised against human megakaryocytic cells that specifically recognizes the cell surface receptor (c-Mpl) for Thrombopoietin (TPO), shows agonist activity. (Deng B., et al., Blood, 92(6):1981-1988 (1998)).

Not all MAbs that are directed to the hormone binding site and act as full competitors for hormone binding are able to act as an agonist and elicit a signal. (Rowlinson S W, et al., 1998 J Biol Chem 273:5307-5314). A restriction in agonism to a narrow range of MAbs has also been reported for the erythropoietin receptor, where an extensive study showed that of 96 MAbs to the receptor, only four possessed agonist activity. (Elliott S, et al., 1996 J Biol Chem 271:24691-24697).

The erbB2 oncogene encodes a growth factor receptor. The overexpression of erbB2 has been correlated with more aggressive tumors and a poorer prognosis. Some antibodies directed to this molecule have an antitumor effect in vivo, but some antibodies do not. (Wang et al. Mol Immunol 2004 February; 40(13):963-969).

Several antibodies used to inhibit tumor necrosis factor (TNF) function work by binding TNF in ways that affect different functions. INFLIXIMAB® neutralizes the biological activity of TNFα by binding with high affinity to the soluble (free floating in the blood) and transmembrane (located on the outer membranes of T cells and similar immune cells) forms of TNFα and inhibits or prevents the effective binding of TNFα with its receptors. REMICADE® and HUMIRA® (another TNF antagonist) are in the subclass of “anti-TNF antibodies” (they are in the form of naturally occurring antibodies), and are capable of neutralizing all forms (extracellular, transmembrane, and receptor-bound) of TNFα. (Choy E H et al. N Engl J Med. 2001; 344:907-916). ENBREL®, a third TNF antagonist, is in a different subclass (receptor-construct fusion protein), and, because of its modified form, cannot neutralize receptor-bound TNFα.

CD28 is present on the surface of T cells and plays an important role in their activation. Signal transduction occurs through CD28 after it is activated (triggered) by binding to its ligand. CD28 activation is dependent on phosphorylation of its cytoplasmic domain. CD28 does not have intrinsic phosphorylation activity but instead is dependent on an extrinsic kinase, e.g. p56lck. However, some antibodies are capable of being superagonists of CD28 receptors by preferentially excluding phosphatases (as opposed to kinases) from the vicinity of the receptor.

The high-throughput identification and classification of natural antibodies against different epitopes enabled by the ARA platform of the present invention allow simultaneous identification of large numbers of families of antibodies effective in modulating different functions corresponding to different epitope clusters. The methods also enable identification of cryptic epitopes. Modulation of the function of some of the cryptic epitopes identified may also exhibit synergistic effects with the modulation of functions of known functional epitopes or the entire target.

In one embodiment, monoclonal antibodies can be arrayed on a solid surface and grouped by clone in discrete target-specific elements in an ARA. In some embodiments the MAbs are immobilized on the internal surface of a vessel selected from the group consisting of a microtiter well, microtiter plate, test tube, Petri dish, microfluidic channel, and microarray. The antibodies can then be tested in situ for ability to elicit a signal from an appropriate reporter cell. Generally, in a preferred embodiment of the methods herein, the antibody is non-diffusably bound to an insoluble support having isolated sample receiving areas (e.g. a microtiter plate, an array, etc.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon®, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is not diffusible.

A cell line that has been engineered to directly or indirectly express a measurable “reporter” substance (detectable label) in response to modulation of the activity of a cell-surface receptor can be used to screen for monoclonal antibodies that activate or inhibit that receptor. In some embodiments the activation of the cell surface molecule (e.g., receptor) is coupled to the activity of an enzyme capable of effecting the cleavage of a covalent bond of a substrate. The enzyme may be selected from the group consisting of β-lactamase, α-galactosidase, β-galactosidase, α-glucosidase, β-glucosidase, α-mannosidase, β-mannosidase, acid phosphatase, alkaline phosphatase and phosphodiesterase II. The substrate may be selected from the group consisting of p-aminophenyl-β-D-galactopyranoside, p-aminophenyl-α-D-galactopyranoside, p-aminophenyl-α-D-glucopyranoside, p-aminophenyl-β-D-glucopyranoside, p-aminophenyl-α-D-mannopyranoside, p-aminophenyl-β-D-mannopyranoside, p-aminophenylphosphate, and p-aminophenylphosphorylcholine or derivative thereof. Cleavage of the substrate is typically linked to a detectable calorimetric or fluorimetric reaction.

In some embodiments, the detectable label is a fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, and enzyme that yield a magnetic product. In very particular embodiments, the detectable label is ruthenium or multiple ruthenium labels.

Screening of cells treated with dyes and fluorescent reagents is well known in the art. There is a considerable body of literature related to genetic engineering of cells to produce fluorescent proteins, such as modified green fluorescent protein (GFP), as a reporter molecule. Some properties of wild-type GFP are disclosed by Morise et al. (Biochemistry 13 (1974), p. 2656-2662), and Ward et al. (Photochem. Photobiol. 31 (1980), p. 611-615). The GFP of the jellyfish Aequorea victoria has an excitation maximum at 395 nm and an emission maximum at 510 nm, and does not require an exogenous factor for fluorescence activity. Luminogenic detectable substrates such as luciferase may also be employed.

U.S. Pat. Nos. 5,401,629 and 5,436,128 describe assays and compositions for detecting and evaluating the intracellular transduction of an extracellular signal using recombinant cells that express cell surface receptors and contain reporter gene constructs that include transcriptional regulatory elements that are responsive to the activity of cell surface receptors.

Standard high throughput screens (“HTS”) use mixtures of compounds and biological reagents along with some indicator compound loaded into arrays of wells in standard microtiter plates with 96 or 384 wells. The signal measured from each well, either fluorescence emission, optical density, or radioactivity, integrates the signal from all the material in the well giving an overall population average of all the molecules in the well. Science Applications International Corporation (SAIC) 130 Fifth Avenue, Seattle, Wash. 98109) describes an imaging plate reader. This system uses a CCD camera to image the whole area of a 96 well plate. The image is analyzed to calculate the total fluorescence per well for all the material in the well. Molecular Devices, Inc. (Sunnyvale, Calif.) describes a system (FLIPR) which uses low angle laser scanning illumination and a mask to selectively excite fluorescence within approximately 200 microns of the bottoms of the wells in standard 96 well plates in order to reduce background when imaging cell monolayers. This system uses a CCD camera to image the whole area of the plate bottom. Although this system measures signals originating from a cell monolayer at the bottom of the well, the signal measured is averaged over the area of the well and is therefore still considered a measurement of the average response of a population of cells. The image is analyzed to calculate the total fluorescence per well for cell-based assays. Fluid delivery devices have also been incorporated into cell based screening systems, such as the FLIPR system, in order to initiate a response, which is then observed as a whole well population average response using a macro-imaging system.

In contrast to high throughput screens, various high-content screens (“HCS”) have been developed to address the need for more detailed information about the temporal-spatial dynamics of cell constituents and processes. High-content screens automate the extraction of multicolor fluorescence information derived from specific fluorescence-based reagents incorporated into cells (Giuliano and Taylor (1995), Curr. Op. Cell Biol. 7:4; Giuliano et al. (1995) Ann. Rev. Biophys. Biomol. Struct. 24:405). Cells are analyzed using an optical system that can measure spatial, as well as temporal dynamics. (Farkas et al. (1993) Ann. Rev. Physiol. 55:785; Giuliano et al. (1990) In Optical Microscopy for Biology. B. Herman and K. Jacobson (eds.), pp. 543-557. Wiley-Liss, New York; Hahn et al (1992) Nature 359:736; Waggoner et al. (1996) Hum. Pathol. 27:494).

High-content screens can be performed on either fixed cells, using fluorescently labeled antibodies, biological ligands, and/or nucleic acid hybridization probes, or live cells using multicolor fluorescent indicators and “biosensors.” The choice of fixed or live cell screens depends on the specific cell-based assay required.

Fixed cell assays are the simplest, since an array of initially living cells in a microtiter plate format can be treated with various compounds and doses being tested, then the cells can be fixed, labeled with specific reagents, and measured. No environmental control of the cells is required after fixation. Spatial information is acquired, but only at one time point. The availability of thousands of antibodies, ligands and nucleic acid hybridization probes that can be applied to cells makes this an attractive approach for many types of cell-based screens. The fixation and labeling steps can be automated, allowing efficient processing of assays.

Live cell assays are more sophisticated and powerful, since an array of living cells containing the desired reagents can be screened over time, as well as space. Environmental control of the cells (temperature, humidity, and carbon dioxide) is required during measurement, since the physiological health of the cells must be maintained for multiple fluorescence measurements over time. There is a growing list of fluorescent physiological indicators and “biosensors” that can report changes in biochemical and molecular activities within cells (Giuliano et al., (1995) Ann. Rev. Biophys. Biomol. Struct. 24:405; Hahn et al., (1993) In Fluorescent and Luminescent Probes for Biological Activity. W. T. Mason, (ed.), pp. 349-359, Academic Press, San Diego).

The availability and use of fluorescence-based reagents has helped to advance the development of both fixed and live cell high-content screens. Advances in instrumentation to automatically extract multicolor, high-content information has recently made it possible to develop HCS into an automated tool. An article by Taylor, et al. (American Scientist 80 (1992), p. 322-335) describes many of these methods and their applications.

In a typical assay, cells expressing a target receptor and engineered to comprise a detectable reporter gene system sensitive to activation or inhibition of the receptor are used to contact an ARA comprising monoclonal antibodies directed to the receptor molecule. In some embodiments, the ARA comprises a multi-well (96 or 384 well) format wherein each well comprises a known monoclonal antibody. It may be necessary to provide a plurality of “spots” comprising a single MAb such that a sufficient concentration of MAb is present to elicit a detectable signal. Likewise, up to 10³, 10⁴, or 10⁵ cells per well may be necessary to elicit a signal. In some embodiments, a cell culture chip for real-time monitoring of cell cultures in micro scales as described in U.S. Pub. Pat App. No. 20070275435 may be used.

After reporter cells are allowed to contact the surface of an ARA device, cells that are not captured by the test monoclonal antibodies grouped in discrete elements on the device are removed by applying a fluid shear force. Captured cells are then cultured on the device in a manner that permits cell growth and the expression of the reporter substance. The reporter substance, which is retained within the captured cells, is then measured directly on the ARA device (e.g. by use of a detectable substrate). In this way monoclonal antibodies that have receptor-agonist or receptor-antagonist activity can be identified by presence or absence of reporter signal elicited from cells captured on discrete elements of the ARA device that represent groups of individual monoclonal antibodies.

For example, NF-κB (nuclear factor kappa-light-chain-enhancer of activated B cells) is a protein complex that acts as a transcription factor. NF-κB is found in almost all animal cell types and is involved in cellular responses to stimuli. Stimulation of a wide variety of cell-surface receptors, such as RANK, TNFR, leads directly to NF-κB activation and fairly rapid changes in gene expression. A human embryonic kidney cell line that stably expresses the beta-lactamase gene under the regulation of an NF-κB response element (NF-κB-bla HEK 293T CellSensor Cell Line, Invitrogen Corp., Calif.) responds to stimulation with Tumor Necrosis Factor-alpha (TNFa) leading to activation of the NF-κB signaling pathway and subsequent beta-lactamase expression. Expression of beta-lactamase is quantified using a fluorescence resonance energy transfer (FRET)-based substrate (LiveBLAzer-FRET B/G Substrate, Invitrogen Inc., Calif.). The substrate is a lipophilic, esterified compound that readily enters the reporter cell line. Upon cleavage by endogenous cytoplasmic esterases, the substrate is converted into a negatively charged substrate that is retained in the cytosol. Beta-lactamase cleavage spatially separates the two chromophors of the substrate disrupting FRET and produces a blue fluorescence signal at 450 nm (upon excitation at 409 nm). In the absence of beta-lactamase cleavage, the substrate produces a green fluorescence signal at 520 nm (upon excitation at 409 nm). The ratio of blue to green fluorescence increases with increasing beta-lactamase activity.

Stimulation of Toll-like receptors (TLRs) leads to activation of NF-κB. (Hayden M S, West A P, Ghosh S (October 2006). “NF-κB and the immune response”. Oncogene 25 (51): 6758-6780). Receptor agonist activity in a monoclonal antibody against TLR may lead to a higher level of endogenous NF-κB activation by TNFa and subsequent increased beta-lactamase expression in the reporter cell line. Conversely, antagonist activity may lead to a decrease in beta-lactamase expression. The degree of TLR modulation in the presence of a monoclonal antibody against TLR may be determined by monitoring a change in the ratio of blue to green fluorescence signal produced by the FRET substrate, e.g., an increase in the ratio of blue to green fluorescence signal is indicative of a TLR activator and a decrease in the ratio blue to green fluorescence signal is indicative of a TLR inhibitor. The degree of TLR activity in the presence of a given MAb may be compared to the level of TLR activity in a control (e.g., in the presence of a compound having known activity).

The methods of the invention also relate to therapeutic antibodies that can be generated from recovered V gene sequences and that are directed to different functional epitopes of a target pathogen or antigen, or that have functional effects on a target receptor.

The methods of the present invention can also be applied to screening small molecules. By identifying epitopes and epitope clusters associated with a specific function, synthetic and natural small molecule products can be tested for effectiveness and ability to bind to functional epitopes identified by methods of the invention.

The methods of the invention also relate to vaccine design by identifying different epitope clusters and enabling the preparation of vaccines directed to different parts of a target pathogen or antigen.

The methods of the invention also relate to therapeutic antibodies that can be generated from recovered V gene sequences and.

### Kits

The present invention provides kits that can be used in the above methods. In one embodiment, a kit comprises an array (ARA) comprising antibodies of the invention, preferably a purified antibody, in one or more containers. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

A kit may also include a non-attached reporter-labeled anti-human antibody. In this embodiment, binding of the antibody to the polypeptide antigen can be detected by binding of the said reporter-labeled antibody. Cell lines comprising a reporter system coupled to a function of a protein with an extracellular domain (e.g., a receptor) are included in some kits. Colorimetric, or fluorimetric or luminometric detection reagents are included in some kits

In another embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In one embodiment, the invention includes a diagnostic kit for use in screening serum containing antigens of the polypeptide of the invention. The diagnostic kit includes a substantially isolated antibody array specifically immunoreactive with polypeptide or polynucleotide antigens, and means for detecting the binding of the polynucleotide or polypeptide antigen to the antibody.

Thus, the invention provides an assay system or kit for carrying out this diagnostic method. The kit generally includes a support with surface-bound recombinant antigens, and a reporter-labeled anti-human antibody for detecting surface-bound anti-antigen antibody.

All publications and patent applications cited in this specification are herein incorporated by reference as if each individual publication or patent application is specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for producing an antibody repertoire array (ARA), the method comprising:
(a) obtaining at least 10⁴ memory B-cells from each of an effective number of human donors;
(b) preparing a population of human B-cells, wherein said population contains at least 10⁵ different species of naturally occurring antibodies wherein each of the antibodies has naturally paired heavy and light chains;
(c) dividing said population of B cells into subpopulations of B cells wherein each subpopulation produces at least 1 different species of antibody;
(d) expanding each subpopulation of B cells to produce expanded B-cell cultures;
(e) culturing each of said B-cell cultures in culture medium under condition in which said B-cells secrete antibodies into said culture medium; and
(f) disposing said antibodies secreted into the culture medium onto a solid surface, thereby producing an antibody repertoire array (ARA) comprising an antibody library.

2. The method of claim 1, further comprising:
(g) interrogating the antibody repertoire array with a target to identify an antibody or antibody variable region or a portion thereof that is specific for said target.

3. The method of claim 1, wherein the B cells are immortalized to produce an immortalized B-cell culture.

4. The method of claim 1, further comprising the steps of
(h) determining which B-cell culture produces said target antibody; and
(i) isolating the B-cell which produces said target antibody from said B-cell culture.

5. The method of claim 1, wherein the antibodies are disposed on a surface of the array wherein said surface comprises Protein A or Protein G which in turn captures the Fc regions of the antibodies.

6. The method of claim 1, wherein the B cells in step (b) are disposed in wells in microtiter plates.

7. The method of claim 1, wherein the population of B-cells in step (b) comprises at least 10⁷ different species of naturally occurring antibodies.

8. The method of claim 1, wherein said effective number of human donors is at least 10.

9. The method of claim 1, wherein the antibody library comprises at least 10⁵ naturally occurring human antibodies having naturally paired V_{H} and V_{L} regions, wherein said antibodies have been secreted from immortalized human B-cells that were obtained from a sufficiently diverse patient population such that the antibodies in said library have a diversity of binding activities substantially similar to the entire human immunome.

10. The method of claim 9, wherein the naturally occurring human antibodies are expressed from human B cells that recognize at least 10² different targets.

11. The method of claim 9, further wherein the B cells are immortalized.

12. The method of claim 11, wherein the immortalized B cells express Epstein-Barr Virus antigens.

13. The method of claim 11, wherein the immortalized B cell secrete antibodies against a pathogen selected from the group consisting of: a RNA virus, a DNA virus, a bacterium, an yeast, a parasite, and a fungus.

14. The method of claim 11, wherein the immortalized B cell secrete antibodies against an antigen expressed by a malignant or benign tumor cell.

15. The method of claim 11, wherein the immortalized B cell secrete antibodies against an antigen selected from the group consisting of: a polypeptide associated with a neurodegenerative disease; a cytokine, a chemokine, a growth factor, an adhesion molecule, and a co-stimulatory molecule, and receptors thereof.

16. A method for making naturally paired immunoglobulins, the method comprising the steps of:
(a) isolating RNA samples from non-immortalized B-cell populations each expressing on average 1-100 different species of antibodies;
(b) performing reverse transcriptase-polymerase chain reaction (RT-PCR) on a plurality of the RNA samples; and
(c) isolating DNA corresponding to V_{H} and V_{L} regions capable of natural pairing.
(d) cloning said DNA corresponding to V_{H} and V_{L} regions in a suitable host capable of expression of said V_{H} and V_{L} regions; and
(e) expressing said V_{H} and V_{L} regions in the context of an immunoglobulin heavy and light chain, such that a naturally paired immunoglobulin is formed.

17. The method of claim 16, wherein non-immortalized B-cell populations each expressing on average 1-100 different species of antibodies are prepared by a method comprising:
(a) obtaining at least 10⁴ memory B-cells from each of an effective number of human donors;
(b) preparing a population of human B-cells, wherein said population contains at least 10⁵ different species of naturally occurring antibodies wherein each of the antibodies has naturally paired heavy and light chains;
(c) dividing said population of B cells into subpopulations of B cells each subpopulation produces on average 1-100 different species of antibodies;
(d) optionally, expanding each subpopulation of B cells to produce an expanded B-cell culture; and
(e) storing each sub-population under conditions suitable for preserving its RNA content,
wherein a library of non-immortalized B-cell populations each expressing on average 1-100 different species of antibodies is produced.

18. A method for making a target specific antibody, the method comprising:
(a) obtaining B-cells from human donors previously exposed to the target, wherein said B-cell population contains at least 10⁵ different species of naturally occurring antibodies with naturally paired heavy and light chains;
(b) dividing said population of B cells into subpopulations of B cells wherein each subpopulation produces on average 1-100 different species of antibodies;
(c) expanding each subpopulation of B cells to produce expanded B-cell cultures under conditions in which said B-cells secrete antibodies into said culture medium;
(d) disposing said antibodies secreted into the culture medium from each of said B-cell cultures at distinct locations on a solid surface to create an antibody repertoire array (ARA); and
(e) interrogating the antibody repertoire array with a native target molecule to identify one or more antibody populations that is specific for said target.

19. The method of claim 18, further comprising the steps of:
(f) preparing RNA samples from each of said B-cell cultures corresponding to an antibody populations that is specific for said target;
(g) performing reverse transcriptase-polymerase chain reaction (RT-PCR) on a plurality of the RNA samples;
(h) isolating DNA corresponding to V_{H} and V_{L} regions capable of natural pairing;
(i) cloning said DNA corresponding to V_{H} and V_{L} regions in a suitable host capable of expression of said V_{H} and V_{L} regions; and
(j) expressing said V_{H} and V_{L} regions in the context of an immunoglobulin heavy and light chain, such that a naturally paired immunoglobulin is formed.

20. The method of claim 19, wherein the target is a virus, bacteria, an yeast, a parasite, a fungus, or other pathogen.

21. The method of claim 20, wherein the target is human immunodeficiency virus (HIV).

22. The method of claims 20, wherein the native target molecule is a virion, a virus like particle, a virus infected cell, a viral protein, or a fragment thereof.

23. The method of claim 18, further comprising the step of: identifying cross-reactive antibodies, wherein the target comprises a plurality of targets comprising multiple species of targets or a plurality of serotypes of the same target.

24. A method for screening antibodies based on epitope clustering, the method comprising:
(a) providing a gene fragment phage display (GFPD) library generated from gene fragments representing parts of a target protein, wherein the GFPD library members are clustered according to correspondence with one or more epitopes;
(b) providing an intact target protein;
(c) providing an antibody repertoire array (ARA) generated according to claim 1 from blood samples of subjects with prior exposure to amounts of a target sufficient to mount an immune response;
(d) interrogating the ARA with the intact target and/or epitope-specific clusters of GFPD library members derived from the target; and
(e) identifying one or more antibody populations that is specific for said intact target and at least one epitope cluster.

25. The method of claim 24, further comprising the steps of:
(f) preparing RNA samples from each of said B-cell cultures corresponding to an antibody population that is specific for said epitope cluster;
(g) performing reverse transcriptase-polymerase chain reaction (RT-PCR) on a plurality of the RNA samples;
(h) isolating DNA corresponding to V_{H} and V_{L} regions capable of natural pairing.
(i) cloning said DNA corresponding to V_{H} and V_{L} regions in a suitable host capable of expression of said V_{H} and V_{L} regions; and
(j) expressing said V_{H} and V_{L} regions in the context of immunoglobulin heavy and light chains, such that a naturally paired immunoglobulin is formed.

26. The method of claim 24, further comprising: identifying a new epitope based on the pattern of recognition of the ARA by the intact target and the GFPD library members.

27. The method of claim 24, wherein the GFPD library members are clustered according to correspondence with one or more epitopes by a method comprising:
providing a gene encoding a target protein;
fragmenting said gene into gene fragments;
preparing a phage display library comprising the GFPD library members;
panning the GFPD library on antibodies specific for the target; and
grouping each GFPD library member according to correspondence with one or more clusters.

28. The method of claim 27, further comprising grouping GFPD library members overlaying the GFPD library members on a known three dimensional structure of the target.

29. The method of claim 24, further comprising testing for a synergism between functions of two or more epitope clusters by:
preparing a first naturally paired immunoglobulin formed by expressing V_{H} and V_{L} regions sequenced from an antibody population that is specific for an epitope cluster;
preparing a second naturally paired immunoglobulin formed by expressing V_{H} and V_{L} regions sequenced from an antibody population that is specific for a different epitope cluster;
administering both first and second naturally paired immunoglobulins individually and in combination to a test system for measuring activity of the intact target; and
determining an activity or a synergy of activities of the new epitope that is related to the known function.

30. A vaccine preparation, comprising antibodies effective against a functional epitope cluster determined by the method of claim 24.

31. A therapeutic antibody preparation, comprising antibodies effective in modulating a function of the target associated with one or more epitope clusters determined by the method of claim 24.

32. A method for screening monoclonal antibodies for the presence of a biological function related to a target molecule that is present on a cell surface, the method comprising:
providing an antibody repertoire array (ARA) generated according to claim 1, the ARA comprising a plurality of monoclonal antibodies located at discrete locations on a surface, wherein the antibodies are directed against a specific target molecule that is present on a cell surface;
contacting the ARA with cells comprising the specific target molecule that is present on the cell surface; and
identifying those monoclonal antibodies which have an inhibiting or activating effect on the specific target molecule that is present on the cell surface.

33. The method of claim 32, further comprising:
contacting the ARA with reporter cells, wherein the reporter cells have been engineered to express a detectable signal when contacted with an agonist or antagonist of the cell surface target molecule present on the surface of the reporter cell;
incubating the reporter cell with the monoclonal antibodies in the presence of a substrate necessary for generating a detectable signal, wherein a change in level of the detectable signal indicates the presence of a cell surface target molecule antagonist or agonist function of the monoclonal antibody.

34. The method of claim 32, wherein the specific target molecule that is present on the cell surface is a receptor molecule.

35. The method of claim 34, wherein the receptor is selected from the group consisting of: peripheral membrane protein receptors, transmembrane receptors, metabotropic receptors, G protein-coupled receptors (GPCRs), receptor tyrosine kinases, guanylyl cyclase receptors, ionotropic receptors responsive to extracellular ligands, receptor tyrosine kinases, cytokine receptors, receptor guanylyl cyclases, receptor serine/threonine protein kinases, insulin receptor, insulin-like growth factor receptor, human growth hormone receptor, glucose transporters, transferrin receptor, epidermal growth factor receptor, low density lipoprotein receptor, leptin receptor, interleukin receptors, IL-1 receptor, IL-2 receptor, muscarinic acetylcholine receptor, adenosine receptors, adrenoceptors, gaba receptors, angiotensin receptors, cannabinoid receptors, cholecystokinin receptors, dopamine receptor, glucagon receptors, metabotropic glutamate receptors, histamine receptors, olfactory receptors, opioid receptors, rhodopsin, secretin receptors, serotonin receptors, somatostatin receptors, calcium-sensing receptors, growth factor receptors, co-stimulatory factor receptors, protease-activated receptors, T cell receptors, B cell receptors, ITIM-containing receptors, ITAM-containing receptors, members of the TNFR superfamily, members of the TNF superfamily, ion channels, and chemokine receptors.

36. The method of claim 35 wherein the antibody functions as a full agonist, partial agonist, antagonist or inverse agonist of the receptor protein.

37. The method of claim 32, wherein the detectable signal is fluorophore, chemical dye, radioactive binding agent, chemiluminescent binding agent, electrochemiluminescent agent, magnetic binding agent, paramagnetic binding agent, promagnetic binding agent, enzyme that yield a colored product, enzyme that yield a chemiluminescent product, enzyme that yields a magnetic product or ruthenium.

38. The method of claim 32, wherein the activation of the cell surface molecule is coupled to an intracellular signaling pathway linked to an activity of an enzyme capable of effecting a substrate.

39. The method of claim 38, wherein the enzyme is selected from the group consisting of β-lactamase, α-galactosidase, β-galactosidase, α-glucosidase, β-glucosidase, α-mannosidase, β-mannosidase, acid phosphatase, alkaline phosphatase and phosphodiesterase II.

40. The method of claim 38, wherein the substrate is selected from the group consisting of p-aminophenyl-β-D-galactopyranoside, p-aminophenyl-α-D-galactopyranoside, p-aminophenyl-α-D-glucopyranoside, p-aminophenyl-β-D-glucopyranoside, p-aminophenyl-α-D-mannopyranoside, p-aminophenyl-β-D-mannopyranoside, p-aminophenylphosphate, and p-aminophenylphosphorylcholine or derivative thereof.

41. The method of claim 38, wherein the effect of the enzyme on the substrate is couple to a chemical, luminometric, calorimetric or fluorimetric reaction.

42. The method of claim 32, wherein the ARA is arranged in a 96 or 384 well plate, wherein each well comprises monoclonal antibodies from a single B cell clone, and further wherein the concentration of monoclonal antibodies is sufficient to elicit a signal from the cell surface target molecule.

43. The method of claim 42, wherein each well is contacted with greater than about 10³ reporter cells.

44. The method of claim 42, wherein the detectable label is not secreted from the reporter cell.

45. The method of claim 42, wherein the detectable label is secreted from the reporter cell.

46. The method of claim 42 wherein each well is contacted with reporter cells which are incubated under conditions suitable for cell growth until a concentration in the order of greater than about 10³ reporter cells is reached.

47. The method of claim 32, wherein the screening is a high throughput screen.

48. The method of claim 32, wherein the screening is a high-content screen.

49. The method of claim 32, wherein activation of the cell-surface target molecule comprises activation of a signaling pathway which is coupled to 13-lactamase expression.

50. The method of claim 49, wherein expression of 13-lactamase is quantified using a fluorescence resonance energy transfer (FRET)-based substrate.

51. The method of claim 32, wherein the ARA comprises a sufficient concentration of an antibody at each discrete location of the surface to elicit a detectable signal upon contacting the specific target molecule that is present on the cell surface.

52. An antibody repertoire array (ARA) prepared by the method of claim 1.

53. The antibody repertoire array (ARA) of claim 52, wherein the ARA comprises at least 10⁴ human native antibodies expressed from human B cells recognizing at least 10² different targets, each antibody secreted from different B-cells having naturally paired VH and VL chains.

54. The antibody repertoire array (ARA) of claim 52, wherein the antibodies on the ARA recognize at least 10³ different targets.

55. The antibody repertoire array (ARA) of claim 52, wherein the antibodies on the ARA comprises at least 10³ expressed human native antibodies.

56. The antibody repertoire array (ARA) of claim 52, wherein the ARA comprises at least 10⁵ naturally occurring human antibodies having naturally paired V_{H} and V_{L} regions, wherein said antibodies have been secreted from immortalized human B-cells that were obtained from a sufficiently diverse patient population such that the antibodies in said library have a diversity of binding activities substantially similar to the entire human immunome.

57. The antibody repertoire array (ARA) of claim 52, wherein the ARA comprises naturally occurring human antibodies against a pathogen selected from the group consisting of: a RNA virus, a DNA virus, a bacterium, an yeast, a parasite, and a fungus.

58. The antibody repertoire array (ARA) of claim 52, wherein the ARA comprises naturally occurring human antibodies against an antigen expressed by a malignant or benign tumor cell.

59. The antibody repertoire array (ARA) of claim 52, wherein the ARA comprises naturally occurring human antibodies against an antigen selected from the group consisting of: a polypeptide associated with a neurodegenerative disease; a cytokine, a chemokine, a growth factor, an adhesion molecule, and a co-stimulatory molecule, and receptors thereof.

60. The antibody repertoire array (ARA) of claim 52, wherein the ARA comprises naturally occurring human antibodies against an epitope-specific cluster from a gene fragment phage display (GFPD) library representing a target.

61. The antibody repertoire array (ARA) of claim 52, wherein the ARA comprises naturally occurring human antibodies against a target molecules that occur on a cell surface.

62. The antibody repertoire array (ARA) of claim 61, wherein the cell surface molecule is a receptor molecule.
